(19) United States
(12) Patent Application Publication
(10) Pub. No.: US 2019/0328363 A1
(43) Pub. Date: Oct. 31, 2019

(54) ULTRASOUND DIAGNOSTIC APPARATUS AND ULTRASOUND SIGNAL PROCESSING METHOD

(71) Applicant: Konica Minolta, Inc., Tokyo (JP)

(72) Inventor: Masaru FUSE, Osaka (JP)

(21) Appl. No.: 16/373,234

(22) Filed: Apr. 2, 2019

(30) Foreign Application Priority Data
Apr. 25, 2018 (JP) 2018-083920

(51) Int. Cl. A61B 8/08; A61B 8/00

(52) U.S. Cl. CPC A61B 8/485; A61B 8/4488; A61B 8/5246; A61B 8/463; A61B 8/5207

(57) ABSTRACT

An ultrasound diagnostic apparatus to which a probe including a plurality of transducers arranged can be connected, causing the probe to transmit a push wave into a subject to detect propagation velocity of a shear wave, includes: a push wave pulse transmitter that uses a plurality of transmission transducers to transmit a push wave that converges to one or more transmission focus points in the subject; a detection wave pulse transmitter that supplies a detection wave pulse to some or all of the plurality of transducers to cause the plurality of transducers to transmit a detection wave; a displacement detector that detects displacement of a tissue at each of a plurality of observation points; an analysis target determiner that determines an analysis target region; and a propagation information analyzer that calculates the propagation velocity of the shear wave at each observation point.

The entire disclosure of Japanese patent Application No. 2018-083920, filed on Apr. 25, 2018, is incorporated herein by reference in its entirety.

### BACKGROUND

### Technological Field

The present disclosure relates to an ultrasound diagnostic apparatus and an ultrasound signal processing method, and more particularly to an analysis of shear wave propagation velocity in a tissue using a shear wave and measurement of elastic modulus of a tissue.

### Description of the Related Art

An ultrasound diagnostic apparatus is a medical examination apparatus that transmits an ultrasound from transducers that constitute an ultrasound probe to the inside of a subject, receives an ultrasound reflected wave (echo) caused by a difference in acoustic impedance of the subject tissue, and generates and displays an ultrasound tomographic image indicating a structure of an internal tissue of the subject on the basis of an obtained electric signal.

In recent years, tissue elastic modulus measurement applying this ultrasound diagnostic technique (Shear Wave Speed Measurement; SWSM, hereinafter the “ultrasound elastic modulus measurement”) has widely been used for examination. This can non-invasively and easily measure the hardness of a tumor mass found in an organ or a body tissue, and is therefore useful in investigating tumor hardness in cancer screening tests and assessing hepatic fibrosis in examination of liver disease.

In this ultrasound elastic modulus measurement, a region of interest (ROI) in a subject is determined, and a push wave (converged ultrasound or acoustic radiation force impulse (ARFI) in which ultrasound is converged to a specific site in the subject from a plurality of transducers is transmitted. Then, an ultrasound for detection (hereinafter the “detection wave”) is transmitted and the reflected wave is received multiple times. It is possible to calculate propagation velocity of a shear wave generated by acoustic radiation pressure of the push wave by conducting propagation analysis of the shear wave, which represents elastic modulus of a tissue. Accordingly, distribution of tissue elasticity is displayed as an elasticity image, for example (for example, JP 2006-500089 A).

A representative method of shear wave propagation analysis includes a method in which displacement in a direction (hereinafter the “depth direction”) perpendicular to the surface of an ultrasound probe in a subject is detected and the movement velocity in a direction (hereinafter the “horizontal direction”) perpendicular to the depth direction of a displacement peak in a time series is detected as a shear wave velocity. The pressing direction of the push wave is the depth direction. Therefore, the direction of vibrations of the shear wave is the depth direction, and the propagation direction of the shear wave is the horizontal direction. However, because the shear wave propagates roughly radially from the focus point of the push wave, when the degree of matching between the propagation direction and the horizontal direction of the shear wave is low in a portion of a noticed tissue, the shear wave detection precision may be reduced depending on the mismatching.

### SUMMARY

The preset disclosure has been made in view of the aforementioned problem, and it is an object of the present disclosure to increase reliability of elastic modulus measurement results in ultrasound elastic modulus measurement.

To achieve the abovementioned object, according to an aspect of the present invention, there is provided an ultrasound diagnostic apparatus to which a probe including a plurality of transducers arranged can be connected, causing the probe to transmit a push wave in which ultrasound beams are converged into a subject to detect propagation velocity of a shear wave generated by acoustic radiation pressure of the push wave, and the ultrasound diagnostic apparatus reflecting one aspect of the present invention comprises: a push wave pulse transmitter that uses a plurality of transmission transducers selected from the plurality of transducers to transmit a push wave that converges to one or more transmission focus points in the subject; a detection wave pulse transmitter that supplies a detection wave pulse to some or all of the plurality of transducers to cause the plurality of transducers to transmit, following transmission of the push wave, a detection wave that passes by a region of interest indicating an analysis target range in the subject multiple times; a displacement detector that detects displacement of a tissue at each of a plurality of observation points in the region of interest on the basis of reflected detection waves received in a time series by the plurality of transducers corresponding to each of detection waves of the multiple times; an analysis target determiner that determines an analysis target region, that is a target of shear wave propagation analysis, on the basis of steepness of a time change of displacement of the tissue at the plurality of observation points; and a propagation information analyzer that calculates the propagation velocity of the shear wave at each observation point present in the analysis target region on the basis of displacement of the tissue at the plurality of observation points present in the analysis target region.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a schematic diagram illustrating an outline of an SWS sequence by an ultrasound elastic modulus measurement method of an ultrasound diagnostic apparatus according to an embodiment;

FIG. 2 is a functional block diagram of an ultrasound diagnostic system including an ultrasound diagnostic apparatus;

FIG. 3A is a schematic diagram illustrating a position of transmission focus point of a push wave generated by a push wave pulse generator;

FIG. 3B is a schematic diagram illustrating a configuration outline of a detection wave pulse generated by a detection wave pulse generator;

FIG. 4A is a functional block diagram illustrating a configuration of a transmitter;

FIG. 4B is a functional block diagram illustrating a configuration of a detection wave receiver;

FIG. 5A is a schematic diagram illustrating an outline of push wave transmission;

FIG. 5B is a schematic diagram illustrating an example of a push wave pulse;

FIG. 6A is a schematic diagram illustrating an outline of detection wave transmission;

FIG. 6B is a schematic diagram illustrating an outline of reflected detection wave reception;

FIG. 7 is a schematic diagram illustrating an outline of a method of calculating an ultrasound propagation path with a delay processor;

FIG. 8 is a functional block diagram illustrating configurations of a displacement detector, a propagation information analyzer, and an elastic modulus calculator;

FIG. 9 is a schematic diagram illustrating an outline of a process of an integral SWS sequence of an ultrasound diagnostic apparatus;

FIG. 10 is a flowchart indicating an ultrasound elastic modulus calculation operation of an ultrasound diagnostic apparatus;

FIGS. 11A to 11E are schematic diagrams illustrating a state of generating a shear wave using a push wave pulse;

FIG. 12 is a schematic diagram illustrating displacement detection and a shear wave propagation analysis operation;

FIG. 13 is a flowchart indicating a shear wave propagation information analysis operation of an ultrasound diagnostic apparatus;

FIG. 14A is a schematic diagram illustrating a relative relationship between an observation line, a plurality of observation points present thereon, and a shear wave traveling direction;

FIGS. 14B and 14C are graphs indicating a time change of displacement;

FIG. 15A is a schematic diagram illustrating an operation of specifying an observation point at which sharpness of a displacement peak is maximum from a plurality of observation points present on an adjacent observation line with reference to a position of an observation point;

FIG. 15B is a schematic diagram illustrating an operation of specifying an observation point when the number of transmission focus points of push wave is one;

FIG. 15C is a schematic diagram illustrating an operation of specifying an observation point when the number of transmission focus points of push wave is plural;

FIG. 16 is a schematic diagram illustrating details of shear wave velocity analysis; and

FIGS. 17A to 17C are diagrams illustrating display examples of an elasticity image.

### DETAILED DESCRIPTION OF EMBODIMENTS

Hereinafter, one or more embodiments of the present invention will be described with reference to the drawings. However, the scope of the invention is not limited to the disclosed embodiments.

### Embodiment

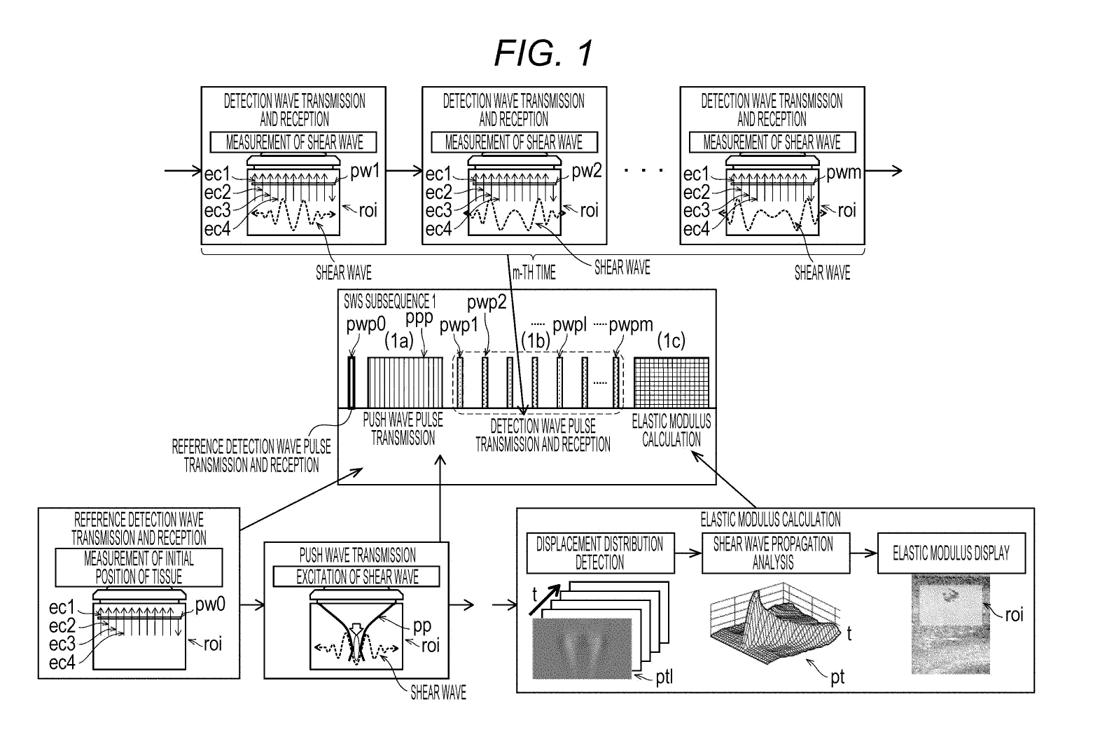

An ultrasound diagnostic apparatus 100 performs processing of calculating shear wave propagation velocity representing tissue elastic modulus according to the ultrasound elastic modulus measurement method. FIG. 1 is a schematic diagram illustrating an outline of a SWS sequence according to the ultrasound elastic modulus measurement method in the ultrasound diagnostic apparatus 100. As illustrated in the middle frame of FIG. 1, the processing of the ultrasound diagnostic apparatus 100 includes the following processes: “reference detection wave pulse transmission and reception”, “push wave pulse transmission”, “detection wave pulse transmission and reception”, and “elastic modulus calculation”.

In the “reference detection wave pulse transmission and reception” process, a reference detection wave pulse pwp0 is transmitted to an ultrasound probe, causing transducers to transmit a detection wave pw0 and receive a reflected wave ec in a range corresponding to a region of interest roi in a subject so as to generate an acoustic line signal, which is reference of the initial position of the tissue.

In the “push wave pulse transmission” process, a push wave pulse ppp is transmitted to the ultrasound probe, causing the transducers to transmit a push wave pp obtained by converging ultrasounds to a specific site in the subject in order to excite a shear wave in the subject tissue.

Then, in the “detection wave pulse transmission and reception” process, a detection wave pulse pwp1 (1 being a natural number from 1 to m, m being the number of times of transmission of a detection wave pulse pwp) is transmitted to the ultrasound probe, causing the transducers to transmit the detection wave pw1 and receive the reflected wave ec multiple times, thereby measuring the shear wave propagation state. In the “elastic modulus calculation” process, first, a tissue displacement distribution pt1 associated with the propagation of the shear wave is calculated in a time series. Next, the shear wave propagation analysis is performed to calculate the propagation velocity of the shear wave indicating a tissue elastic modulus from time series changes of the displacement distribution pt1, and finally the elastic modulus distribution is imaged and displayed as an elasticity image.

The series of processes associated with one-time shear wave excitation based on push wave pp transmission described above is called the “Shear Wave Speed (SWS) sequence”.

<Ultrasound Diagnostic System 1000>

1. Apparatus Outline

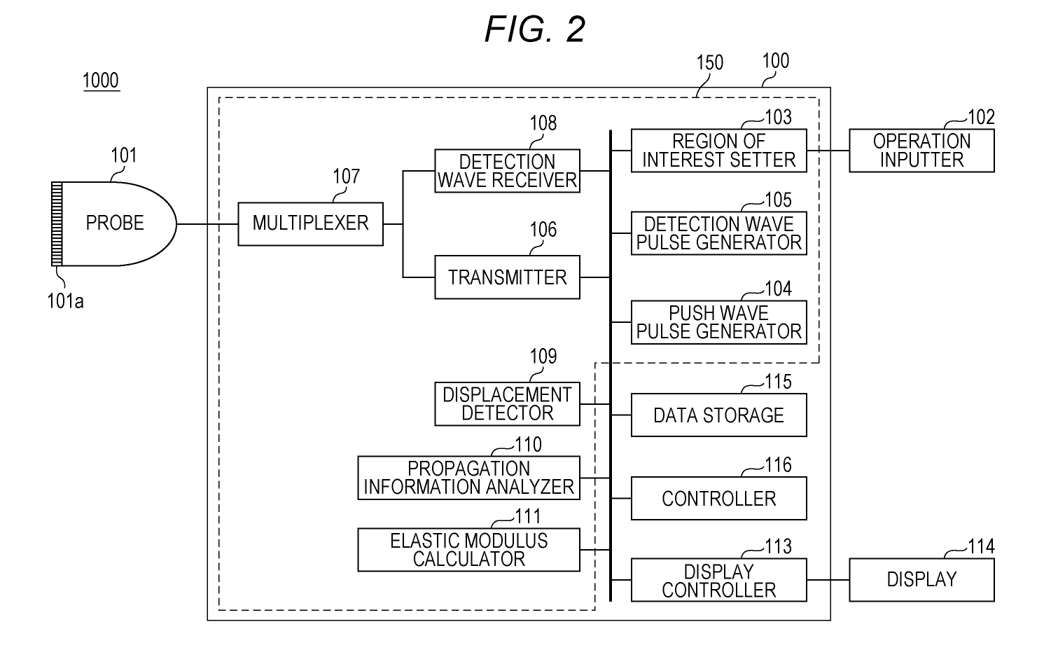

An ultrasound diagnostic system 1000 including the ultrasound diagnostic apparatus 100 according to an embodiment is described with reference to the drawings. FIG. 2 is a function block diagram of the ultrasound diagnostic system 1000 according to an embodiment. As illustrated in FIG. 2, the ultrasound diagnostic system 1000 includes: an ultrasound probe 101 (hereinafter the “probe 101”) in which a plurality of transducers (transducer array) 101a that transmits ultrasounds towards a subject and receives reflected waves are arranged on a front end surface; the ultrasound diagnostic apparatus 100 that causes the probe 101 to transmit and receive ultrasounds and generates an ultrasound signal based on an output signal from the probe 101; an operation inputter 102 that receives an operation input from an examiner; and a display 114 that displays an ultrasound image on a screen. The probe 101, the operation inputter 102, and the display 114 are each configured to be connectable to the ultrasound diagnostic apparatus 100.

Next, each element externally connected to the ultrasound diagnostic apparatus 100 is described.

2. Probe 101

The probe 101 includes a transducer array (101a) including a plurality of transducers 101a arranged, for example, in a one-dimensional direction (hereinafter the “transducer array direction”). The probe 101 converts a pulsed electric signal (hereinafter the “transmission signal”) supplied from a transmitter 106 described later into a pulsed ultrasound. The probe 101, in a state in which a transducer-side outer surface of the probe 101 is in contact with a skin surface of a subject via an ultrasound gel or the like, transmits an ultrasound beam composed of a plurality of ultrasounds emitted from the plurality of transducers towards a measurement target. Then, the probe 101 receives a plurality of reflected detection waves (hereinafter the “reflected wave”) from the subject, converts, by the plurality of transducers 101a, the reflected waves into electrical signals, and supplies the electrical signals to the ultrasound diagnostic apparatus 100.

3. Operation Inputter 102

The operation inputter 102 accepts various operation inputs such as various settings and operations with respect to the ultrasound diagnostic apparatus 100 from an examiner, and outputs the inputs to a controller 116 of the ultrasound diagnostic apparatus 100.

The operation inputter 102 may be, for example, a touch panel integrated with the display 114. In this case, various settings and operations of the ultrasound diagnostic apparatus 100 can be performed through touch operations and drag operations on operation keys displayed on the display 114, and the ultrasound diagnostic apparatus 100 is configured to be operable via the touch panel. Alternatively, the operation inputter 102 may, for example, be a keyboard with various operation keys, various operation buttons, an operation panel with a lever or the like, or a mouse or the like.

4. Display 114

The display 114 is a so-called display device for image display, and displays an image output from a display controller 113 to be described later to a screen. A liquid crystal display, a CRT, an organic EL display, or the like can be used for the display 114.

<Configuration Outline of the Ultrasound Diagnostic Apparatus 100>

Next, the ultrasound diagnostic apparatus 100 according to an embodiment is described.

The ultrasound diagnostic apparatus 100 includes: a multiplexer 107 that selects each transducer to be used for transmission or reception from among the transducers 101a of the probe 101 and secures input and output with respect to the selected transducers; the transmitter 106 that controls timing of high voltage application to each of the transducers 101a of the probe 101 for ultrasound transmission; and the detection wave receiver 108 that performs reception beamforming based on reflected waves received by the probe 101 to generate an acoustic line signal.

Further, the ultrasound diagnostic apparatus 100 includes: a region of interest setter 103 that sets a region of interest roi with reference to the plurality of transducers 101a, the region of interest roi indicating an analysis target range in the subject based on an operation input from the operation inputter 102; a push wave pulse generator 104 that causes the plurality of transducers 101a to transmit a push wave pulse ppp, and a detection wave pulse generator 105 that causes the plurality of transducers 101a to transmit a detection wave pulse pwp1 multiple (m) times after the push pulse ppp.

Further, the ultrasound diagnostic apparatus 100 includes: a displacement detector 109 that detects tissue displacement in the region of interest roi from the acoustic line signal; a propagation information analyzer 110 that performs shear wave propagation information analysis from detected tissue displacement and calculates shear wave wavefront arrival time at each observation point in the region of interest roi to calculate a shear wave propagation velocity, and an elastic modulus calculator 111 that calculates an elastic modulus at each observation point in the region of interest roi.

Further, the ultrasound diagnostic apparatus 100 includes: a data storage 115 that stores an acoustic line signal outputted by the detection wave receiver 108, displacement data outputted by the displacement detector 109, wavefront data, wavefront arrival time data and velocity value data outputted by the propagation information analyzer 110, and elastic modulus data outputted by the elastic modulus calculator 111, and the like; the display controller 113 that forms a display image and causes it to be displayed on the display 114; and the controller 116 that controls each constituent element.

Of these elements, the multiplexer 107, the transmitter 106, the detection wave receiver 108, the region of interest setter 103, the push wave pulse generator 104, the detection wave pulse generator 105, the displacement detector 109, the propagation information analyzer 110, and the elastic modulus calculator 111 constitute an ultrasound signal processing circuit 150.

Elements that constitute the ultrasound signal processing circuit 150, the controller 116, and the display controller 113 are each implemented by a hardware circuit such as Field Programmable Gate Array (FPGA) or an Application Specific Integrated Circuit (ASIC). Alternatively, such elements may be implemented by a programmable device such as a Central Processing Unit (CPU), a General-Purpose computing on Graphics Processing Unit (GPGPU), or a processor, and software. These constituent elements can each be a single circuit component or an aggregate of circuit components. Further, a plurality of constituent elements can be combined into a single circuit component or can be an aggregate of a plurality of circuit components.

The data storage 115 is a computer-readable recording medium, and may be a flexible disk, hard disk, MO, DVD, DVD-RAM, semiconductor memory, or the like. Further, the data storage 115 may be a storage device that is externally connected to the ultrasound diagnostic apparatus 100.

The ultrasound diagnostic apparatus 100 according to a first embodiment is not limited to the ultrasound diagnostic apparatus configured as illustrated in FIG. 1. For example, the multiplexer 107 may be unnecessary, or the transmitter 106 or the detection wave receiver 108, or a portion thereof, may be housed in the probe 101.

<Configuration of Elements of the Ultrasound Diagnostic Apparatus 100>

Next, a configuration of each block included in the ultrasound diagnostic apparatus 100 is described.

1. Region of Interest Setter 103

Generally, when a B mode image, which is a tomographic image of a subject acquired in real time by the probe 101, is being displayed on the display 114, an operator, using the B mode image displayed on the display 114 as an index, specifies an analysis target range in the subject and performs inputting to the operation inputter 102. The region of interest setter 103 sets information specified by the operator from the operation inputter 102 as input, and outputs it to the controller 116. At this time, the region of interest setter 103 may set a region of interest roi that represents an analysis target range in a subject with reference to the position of the transducer array (101a) including the transducers of the probe 101. For example, the region of interest roi may be a whole or partial region of a detection wave radiation region Ax including the transducer array (101a) including the transducers 101a.

2. Push Wave Pulse Generator 104

The push wave pulse generator 104 obtains information indicating the region of interest roi from the controller 116 and sets one or more specific points at a predetermined position near or inside the region of interest roi. Then, by causing the plurality of transducers 101a to transmit a push wave pulse pppₙ (n=1 to nₘₐₓ) from the transmitter 106 one or more times (nₘₐₓ times), the plurality of transducers 101a are caused to transmit a push wave ppₙ (n=1 to nₘₐₓ), which is converged ultrasound beams, to a specific site in the subject corresponding to the specific points (hereinafter the “transmission focus point FPₙ” (n=1 to nₘₐₓ)). Thus, a shear wave is excited in the specific site in the subject. At this time, the number of times of transmission of the push wave pulse pppₙ (nₘₐₓ) may be one to eight. However, the nₘₐₓ is not limited to the above, but may of course be changed as appropriate.

More specifically, the push wave pulse generator 104 determines a position of the transmission focus point FPn of a push wave and a transducer array to transmit the push wave pppₙ (hereinafter the “push wave transmission transducer array Pxn”) on the basis of information indicating the region of interest roi as described below.

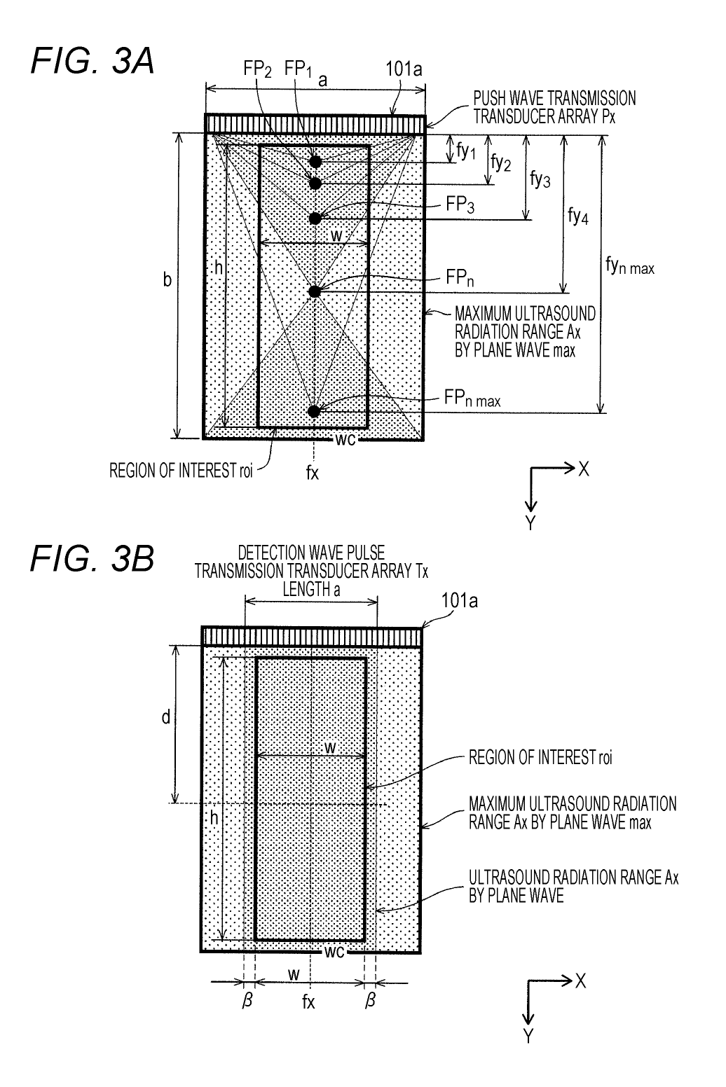

FIG. 3A is a schematic diagram illustrating the position of the transmission focus point FPn of the push wave pppₙ generated by the push wave pulse generator 104. Description is given by way of example in which the array directional length w and the subject depth directional length h of the region of interest roi are equal to or less than the array directional length a and the subject depth directional length b of an ultrasound radiation range of a plane wave, respectively, and the region of interest roi is set near the center of the ultrasound radiation range. In the present embodiment, as illustrated in FIG. 3A, among the positions of the transmission focus point FPn, the array directional transmission focus point position fx, for example, is configured to correspond to an array directional central position we of the region of interest roi.

Further, the push wave transmission transducer array Px is set on the basis of a depth direction transmission focus point position fyn. In the present embodiment, the length of the push wave pulse transmission transducer array Pxn (n=1 to nₘₐₓ) is the length a of the array of all the transducers 101a.

The information indicating the position of the transmission focus point FPₙ and the push wave transmission transducer array Pxₙ is outputted to the transmitter 106 together with a pulse width PWₙ and an application start time PTₙ of the push pulse pppₙ as a transmission control signal. In addition, a time interval PIₙ of the application start time PTₙ may be included. Note that the pulse width PWₙ, the application start time PTₙ, and the time interval PIₙ of the push wave pulse pppₙ will be described later.

Note that the positional relationship between the region of interest roi and the transmission focus point FP is not limited to the above, but may be appropriately changed depending on the form or the like of a portion of the subject to be examined.

For example, the example illustrated in FIG. 3A may be changed to a configuration in which the array direction transmission focus point position fx among the positions of the transmission focus point FP is offset in the positive or negative direction on the x axis from the array direction central position wc of the region of interest roi. In this case, the array direction centers of a region of interest width w and the transducer array are different. Furthermore, the array direction focus point position fx among the transmission focus points FP may be offset in the positive or negative direction on the x axis from the array direction center wc of the region of interest roi so as to be positioned outside the region of interest roi.

In addition, when the region of interest width w is relatively large, push waves in which the array direction transmission focus point position fx of the transmission focus point FPn varies with the transmission focus point FPn may be generated.

In addition, the transmission focus point FP may be set in a predetermined position near the region of interest roi and outside the region of interest roi. At this time, when the transmission focus point FP is set near the region of interest roi, the transmission focus point FP is set at a distance at which a shear wave can reach the region of interest roi with respect to the region of interest roi.

Here, “converging” an ultrasound beam according to a push wave indicates that the ultrasound beam is focused into a focused beam, i.e., an area irradiated by the ultrasound beam decreases after transmission and achieves a minimum value at a specified depth, but is not limited to the case where the ultrasound beam is focused to a single point. In this case, the “transmission focus point FP” indicates an ultrasound beam center at a depth at which an ultrasound beam converges.

In the present specification, hereinafter, the push wave pulse pppₙ, the push wave ppₙ, the push wave transmission transducer array Pxₙ, the transmission focus point FPₙ, the depth direction transmission focus point position fyn, the pulse width PWₙ of the push wave pulse pppₙ, the application start time PTₙ, and the order of transmission (n) of the time interval PIₙ of the application start time PTₙ are described without the letter “n” when they are not distinguished.

3. Detection Wave Pulse Generator 105

The detection wave pulse generator 105 inputs information indicating the region of interest roi from the controller 116 and causes the plurality of transducers 101a belonging to the detection wave pulse transmission transducer array Tx to transmit a detection wave pw such that the transducers 101a transmit the detection wave pulse pwp1 from the transmitter 106 multiple times and an ultrasound beam passes by the region of interest roi. More specifically, based on information indicating the region of interest roi, the detection wave pulse generator 105 determines a transducer array that transmits the detection wave pulse pwp1 (hereinafter the “detection wave transmission transducer array Tx”) such that an ultrasound beam passes by the region of interest roi. At this time, the number of times (m) of transmission of detection wave pulse pwp1 may be, for example, 30 to 100. Furthermore, the transmission interval of the detection wave pulse pwp1 may be, for example, 100 μsec to 150 μsec. However, it is needless to say that the application conditions are not limited to the above, but may be changed as appropriate.

FIG. 3B is a schematic diagram illustrating a configuration outline of the detection wave pulse pwp1 generated by the detection wave pulse generator 105. As illustrated in FIG. 3B, the detection wave pulse generator 105 sets the detection wave pulse transmission transducer array Tx such that a detection wave, a so-called plane wave by which the detection wave pulse transmission transducers are driven in the same phase passes by the entirety of the region of interest roi. The length a of the detection wave pulse transmission transducer array Tx is preferably set to be larger than the region of interest width w. In this example, the region of interest width w is set to be positioned inward, by a predetermined distance β, from an array directional end of the detection wave pulse transmission transducer array Tx. A detection wave pw, which is a plane wave, propagates in a Y direction perpendicular to the transducer array direction. Therefore, the region of interest roi is included in the ultrasound radiation region Ax with a margin of only the distance β at both X-directional ends. Thus, an acoustic line signal can be generated with respect to observation points present across the region of interest roi through one-time detection wave transmission and reception, and the detection wave pulse pwp1 can be transmitted such that an ultrasound beam unfailingly passes by the entirety of the region of interest roi. However, the number of times of detection wave transmission is not limited to the above, but, for example, an acoustic line signal may be generated with respect to an observation point present in part of the region of interest roi through one-time detection wave transmission and reception and the detection wave transmission and reception may be performed multiple times such that acoustic line signals obtained by each transmission and reception may be combined to generate an acoustic line signal with respect to observation points of the entirety of the region of interest roi.

In addition, the detection wave pulse transmission transducer array Tx may be configured to include all of the transducers 101a. The ultrasound radiation region Ax may be a maximum ultrasound radiation region Axₘₐₓ of a plane wave.

The information indicating the detection wave pulse transmission transducer array Tx is outputted to the transmitter 106 as a transmission control signal together with pulse width of the detection wave pulse pwp1.

4. Transmitter 106

The transmitter 106 is a circuit that is connected to the probe 101 via the multiplexer 107, and in order to transmit ultrasounds from the probe 101, controls timing of application of a high voltage to each of the plurality of transducers included in the push wave transmission transducer array Px or the detection wave transmission transducer array Tx, which correspond to all or a portion of the transducers 101a of the probe 101.

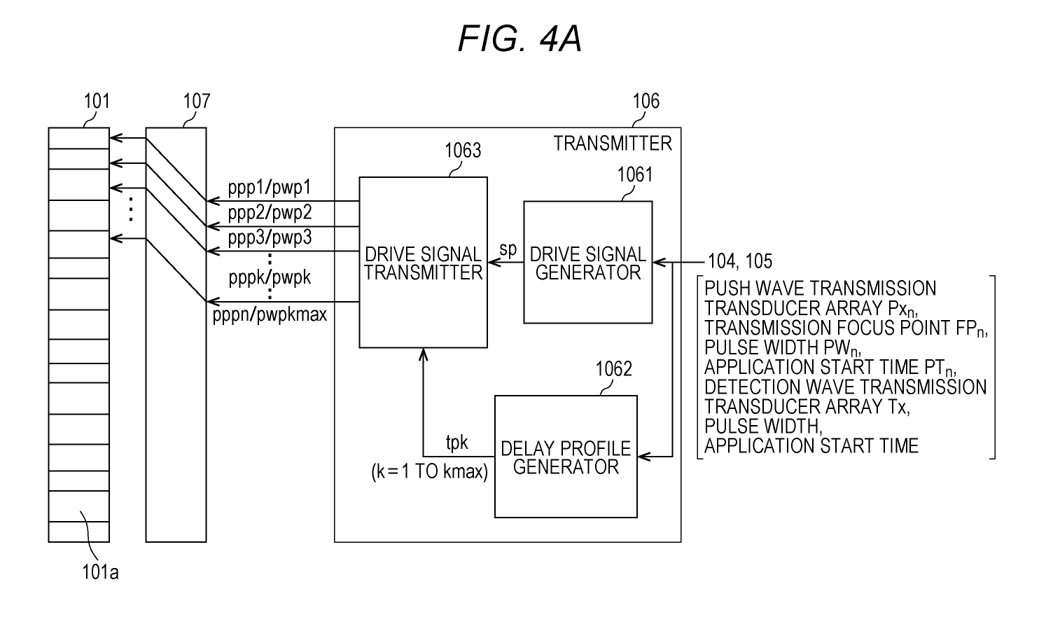

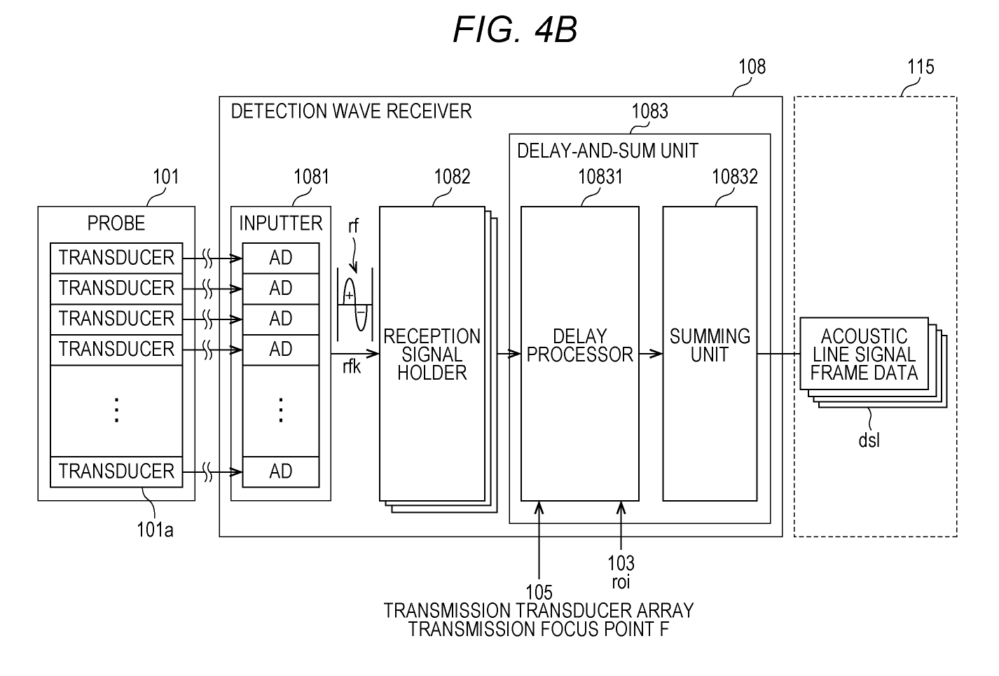

FIG. 4A is a function block diagram illustrating a configuration of the transmitter 106. As illustrated in FIG. 4A, the transmitter 106 includes a drive signal generator 1061, a delay profile generator 1062, and a drive signal transmitter 1063.

(1) Drive Signal Generator 1061

The drive signal generator 1061 is a circuit that generates, among the transmission control signals from the push wave pulse generator 104 or the detection wave pulse generator 105, a pulse signal sp for causing a transmission transducer, which corresponds to all or a portion of the transducers 101a of the probe 101, to transmit an ultrasound beam on the basis of the information indicating the push wave pulse transmission transducer array Px or the detection wave transmission transducer array Tx, the information indicating the pulse width PWn and the application start time PTₙ of the push wave pulse pppₙ, and the information indicating the pulse width and the application start time of the detection wave pulse pwp1.

(2) Delay Profile Generator 1062

The delay profile generator 1062 is a circuit that, among the transmission control signals obtained from the push wave pulse generator 104 or the detection wave pulse generator 105, sets and outputs, with respect to each transducer, a delay time tpk (k being a natural number from 1 to the number of transducers 101a kmax) from the application start time PTₙ that determines a transmission timing of an ultrasound beam on the basis of the information indicating the transmission focus point FPₙ and the push wave transmission transducer array Pxn or the detection wave transmission transducer array Tx. Thus, ultrasound beam focusing is performed by causing the transmission of an ultrasound beam to be delayed for a delay time with respect to each transducer.

(3) Drive Signal Transmitter 1063

The drive signal transmitter 1063 performs push wave transmission processing of supplying the push wave pulse ppp for causing the transducers included in the push wave transmission transducer array Px among the transducers 101a of the probe 101 to transmit a push wave on the basis of a pulse signal sp from the drive signal generator 1061 and a delay time tpk from the delay profile generator 1062. The push wave transmission transducer array Px is selected by the multiplexer 107.

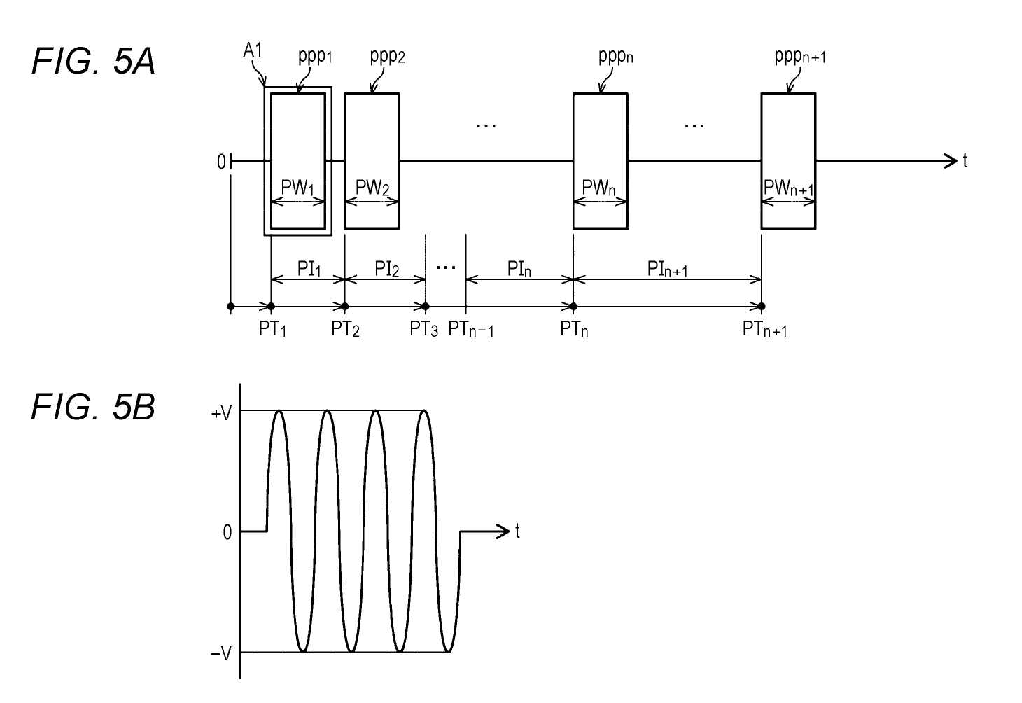

FIGS. 5A and 5B are schematic diagrams illustrating timings of application of a push wave pulse.

A push wave that produces physical displacement in a living body requires a significantly larger power as compared with a transmission pulse used for general B mode display or the like. That is, as a drive voltage to be applied to a puller (ultrasound generator), generally even 30 to 40 V can be acceptable for acquisition of a B mode image, whereas a push wave requires, for example, 50 V or more. In addition, for acquisition of a B mode image, the transmission pulse length is about several μsec, whereas a push wave requires a transmission pulse length of several hundreds of μsec per transmission.

In the present embodiment, as illustrated in FIG. 5A, a push wave pulse pppₙ is transmitted nₘₐₓ times, which is one or more times, to the plurality of transducers 101a from the drive signal transmitter 1063 at each application start time PTₙ. As illustrated in FIG. 5B, the push wave pulse pppₙ includes a burst signal including a predetermined pulse width PWn (time length), a predetermined voltage amplitude (+V to −V), and a predetermined frequency. Specifically, the pulse width PWₙ may, for example, be 100 to 200 μsec, the frequency may, for example, be 6 MHz, and the voltage amplitude may, for example, be +50 V to −50 V. However, of course, the application condition is not limited to the above.

In addition, as illustrated in FIG. 5A, the application start time PTₙ for each push wave pulse pppₙ is configured such that the time interval PIₙ of the application start time PTn for each push wave pulse pppₙ increases in descending order per application of the push wave pulse pppₙ. The pulse width PWₙ for each push wave pulse pppₙ may be constant regardless of the order of application of the push wave pulse pppₙ. Alternatively, the pulse width PWₙ for each push wave pulse pppₙ may increase in descending order per application of the push wave pulse pppₙ.

At the application start time PTn for each push wave pulse pppₙ, a push wave pulse ppp to which a distribution having large delay time tpk is applied is transmitted to the transducer centrally positioned in the transducer array with respect to the push wave transmission transducer array Px. Thus, a push wave ppₙ in which ultrasound beams converge is transmitted from the push wave transmission transducer array Px to a specific site in the subject corresponding to the transmission focus point FPₙ.

In addition, the drive signal transmitter 1063 performs detection wave transmission processing for supplying the detection wave pulse pwp1 that causes each transducer included in the detection wave transmission transducer array Tx among the transducers 101a of the probe 101 to transmit an ultrasound beam. The detection wave transmission transducer array Tx is selected by the multiplexer 107. However, the configuration of supplying the detection wave pulse pwp1 is not limited to the above, but, for example, may not use the multiplexer 107.

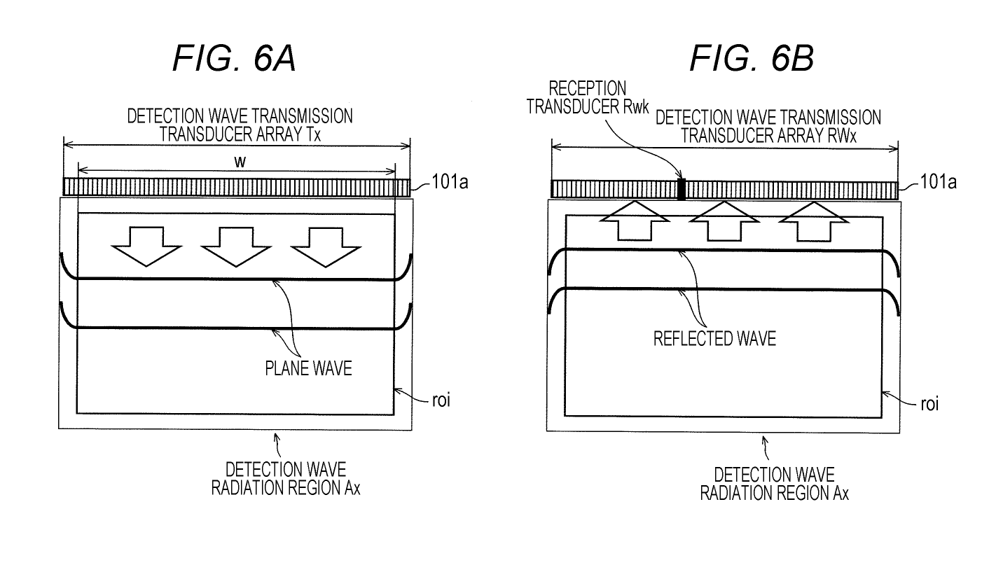

FIG. 6A is a schematic diagram illustrating an outline of detection wave transmission. The delay time tpk is not applied to the transducer included in the detection wave transmission transducer array Tx, and the detection wave pulse pwp1 having an equivalent phase with respect to the detection wave transmission transducer array Tx is transmitted. Thus, as illustrated in FIG. 6A, a plane wave that travels in the subject depth direction is transmitted from each transducer of the detection wave transmission transducer array Tx. A region in a plane corresponding to a range in the subject where the detection wave reaches and including the detection wave transmission transducer array Tx is the detection wave radiation region Ax.

The transmitter 106, after push wave pulse ppp transmission, transmits multiple times the detection wave pulse pwp1 on the basis of the transmission control signal from the detection wave pulse generator 105. After one-time push wave pulse ppp transmission, each time in the series of detection wave pulse pwp1 transmissions performed multiple times from the same detection wave transmission transducer array Tx is called the “transmission event”.

5. Detection Wave Receiver 108

The detection wave receiver 108 is a circuit that, based on reflected waves from a subject tissue received in a time series by the plurality of transducers 101a corresponding to each of the detection wave pulse pwp1 of multiple times, generates an acoustic line signal for a plurality of observation points Pij in the detection wave radiation region Ax to generate a sequence of acoustic line signal frame data ds1 (1 being natural number from 1 to m; referred to as the acoustic line signal frame data ds1 when numbers are not distinguished). That is, the detection wave receiver 108, after detection wave pulse pwp1 transmission, generates an acoustic line signal from an electric signal obtained by the plurality of transducers 101a on the basis of reflected waves received by the probe 101. Here, i is a natural number indicating an x-direction coordinate in the detection wave radiation region Ax, and j is a natural number indicating a y-direction coordinate. Note that an “acoustic line signal” is a signal obtained when delay-and-sum processing is performed on a reception signal (RF signal).

FIG. 4B is a function block diagram illustrating a configuration of the detection wave receiver 108. The detection wave receiver 108 includes an inputter 1081, a reception signal holder 1082, and a delay-and-sum unit 1083.

5.1 Inputter 1081

The inputter 1081 is a circuit that is connected to the probe 101 via the multiplexer 107 and generates a reception signal (RF signal) on the basis of reflected waves at the probe 101. Here, a reception signal rfk (k being a natural number from 1 to n) is a so-called RF signal obtained by A/D converting an electrical signal converted from the reflected wave received by each transducer based on transmission of the detection wave pulse pwp1, and is composed of an array of signals (reception signal array) that is continuous in the transmission direction (subject depth direction) of ultrasounds received by each reception transducer rwk.

The inputter 1081 generates an array of reception signals rfk for each reception transducer rwk with respect to each transmission event on the basis of the reflected waves obtained by each reception transducer rwk. A reception transducer array is composed of a transducer array that is part or all of the transducers 101a of the probe 101, and is selected by the multiplexer 107 on the basis of an instruction from the controller 116. In the present example, all of the transducers 101a are selected as a reception transducer array. This makes it possible to generate a reception transducer array of all transducers by receiving the reflected waves from observation points in the entire area of the detection wave radiation region Ax using all transducers in single reception processing as illustrated in FIG. 6B indicating the outline of the reflected detection wave reception. The generated reception signal rfk is outputted to the reception signal holder 1082.

5.2 Reception Signal Holder 1082

The reception signal holder 1082 is a computer-readable recording medium and may be, for example, a semiconductor memory. The reception signal holder 1082 inputs a reception signal rfk for each reception transducer rwk from the inputter 1081 in synchronization with a transmission event and holds it until one piece of acoustic line signal frame data is generated.

Further, the reception signal holder 1082 may be a part of the data storage 115.

5.3 Delay-and-Sum Unit 1083

The delay-and-sum unit 1083 is a circuit that, in synchronization with a transmission event, after performing delay processing on reception signals rfk received by reception transducers Rpk included in a detection wave pulse reception transducer array Rx from an observation point Pij in the region of interest roi, performs summing for all the reception transducers Rpk to generate an acoustic line signal ds. The detection wave pulse reception transducer array Rx is composed of the reception transducers Rpk that are part or all of the transducers 101a of the probe 101, and is selected by the delay-and-sum unit 1083 and the multiplexer 107 on the basis of an instruction from the controller 116. In the present example, a transducer array including at least all of the transducers constituting the detection wave pulse transmission transducer array Tx for each transmission event is selected as the reflection wave reception transducer array Rx.

The delay-and-sum unit 1083 includes a delay processor 10831 and a summing unit 10832 for processing reception signals rfk.

(1) Delay Processor 10831

The delay processor 10831 is a circuit that compensates reception signals rfk with respect to the reception transducers Rpk in the detection wave pulse reception transducer array Rx according to an arrival time difference (delay) of the reflected ultrasound to each reception transducer Rpk, which is obtained by dividing a difference in distance between the observation point Pij and the reception transducer Rpk by a speed of sound value, and identifies it as a reception signal corresponding to the reception transducer Rpk on the basis of the reflected ultrasound from the observation point Pij.

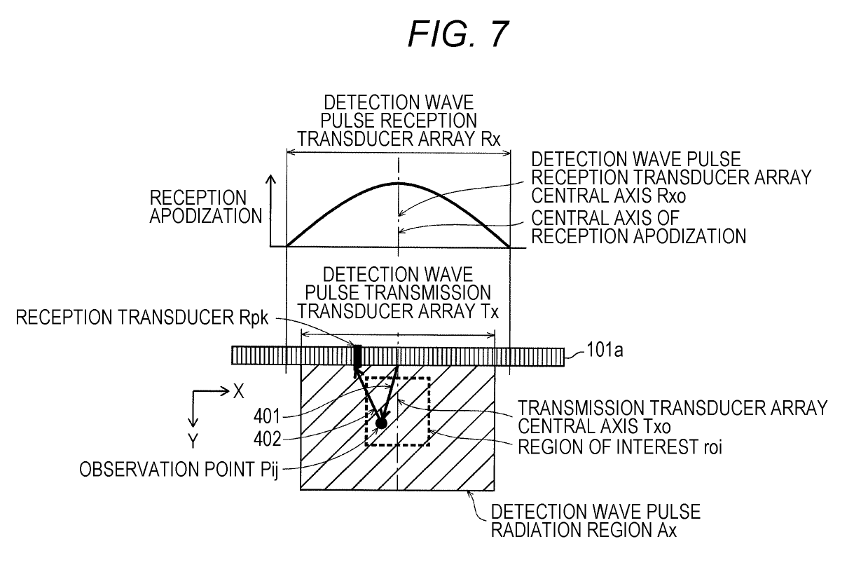

FIG. 7 is a schematic diagram illustrating an outline of an ultrasound propagation path calculation method in the delay processor 10831. FIG. 7 illustrates a propagation path of ultrasounds emitted from the detection wave pulse transmission transducer array Tx, reflected at an observation point Pij at a given position in the region of interest roi, and arrive at the reception transducer Rpk.

a) Calculation of Transmission Time

A detection wave pw1 transmitted from the detection wave transmission transducer array Tx (entirety of transducer array (101a)) is a plane wave as described above. Therefore, the delay processor 10831, in response to a transmission event, calculates a transmission path to the observation point Pij as a shortest path 401 for the detection wave pw1 perpendicularly emitted to the transducer array from the detection wave transmission transducer array Tx to arrive at the observation point Pij, and divides the shortest path by the speed of sound to calculate transmission time.

b) Calculation of Reception Time

The delay processor 10831, in response to a transmission event, regarding the observation point Pij, calculates a reception path for arrival at the reception transducer Rpk included in the detection wave reception transducer array Rx after reflection of the observation point Pij. For the reception path on which the reflected wave at the observation point Pij returns to the reception transducer Rpk, a length of a path 402 from a given observation point Pij to each reception transducer Rpk is calculated geometrically. This is divided by the speed of sound to calculate the reception time.

c) Delay Calculation

Next, the delay processor 10831 calculates total time of propagation to each reception transducer Rpk from the transmission time and the reception time, and calculates a delay to be applied to a reception signal array rfk with respect to each reception transducer Rpk on the basis of the total propagation time.

d) Delay Processing

Next, the delay processor 10831, from the reception signal array rfk corresponding to each reception transducer Rpk, identifies a reception signal rfk corresponding to a delay (reception signal corresponding to the time from which the delay is subtracted) as a signal corresponding to the reception transducer Rpk on the basis of the reflected wave from the observation point Pij.

The delay processor 10831, in response to a transmission event, treats the reception signal rfk from the reception signal holder 1082 as an input, and identifies the reception signal rfk with respect to each reception transducer Rpk for all the observations points Pij positioned in the region of interest roi.

(2) Summing Unit 10832

The summing unit 10832 is a circuit that treats, as an input, the reception signals rfk outputted from the delay processor 10831 and identified corresponding to the reception transducers Rpk and sums it to generate an acoustic line signal dsij subjected to delay-and-sum with respect to the observation point Pij.

Further, the reception signals rfk identified corresponding to each reception transducer Rpk may be multiplied by a reception apodization (weighting sequence) and summed in order to generate an acoustic line signal dsij with respect to the observation point Pij. A reception apodization is a weighting coefficient sequence applied to reception signals corresponding to the reception transducers Rpk in the detection wave reception transducer array Rx. A reception apodization is set such that transducers centrally positioned in the array direction of the detection wave reception transducer array Rx have a maximum weight and the central axis of the distribution of the reception apodization matches a detection wave reception transducer array central axis Rxo, and the distribution has a symmetrical shape with respect to the central axis. The shape of the distribution is not particularly limited.

The summing unit 10832 generates an acoustic line signal dsij for every observation point Pij present in the region of interest roi and generates acoustic line signal frame data ds1.

Then, in synchronization with a transmission event, transmission and reception of the detection wave pulses pwp1 is repeated to generate acoustic line signal frame data ds1 for all transmission events. The generated acoustic line signal frame data ds1 is outputted to and stored in the data storage 115 with respect to each transmission event.

6. Displacement Detector 109

The displacement detector 109 is a circuit that detects displacement of a tissue in the detection wave radiation region Ax from a sequence of the acoustic line signal frame data ds1.

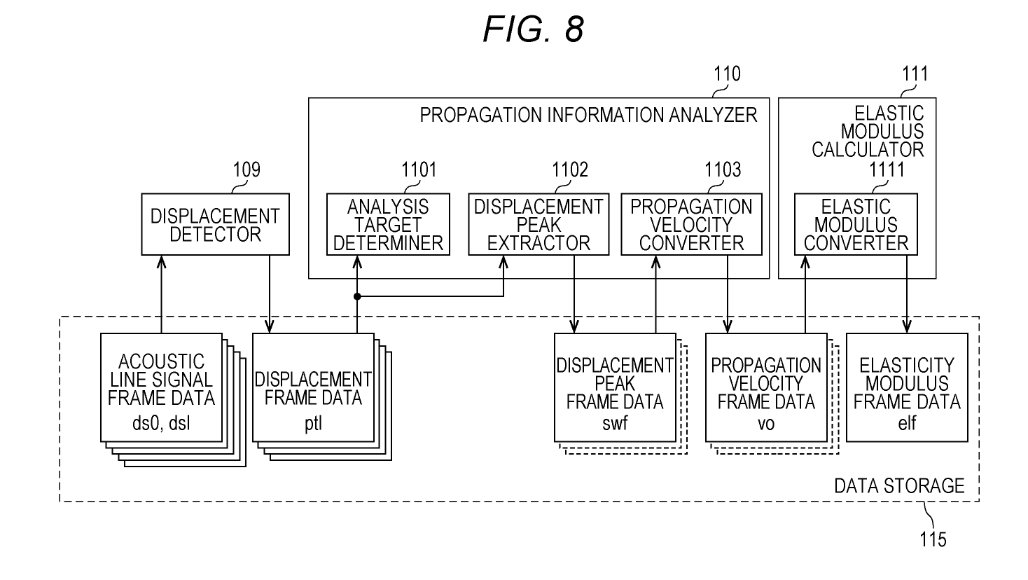

FIG. 8 is a function block diagram illustrating configurations of the displacement detector 109, the propagation information analyzer 110, and the elastic modulus calculator 111.

The displacement detector 109 acquires one frame of acoustic line signal frame data ds1, which is a target of displacement detection included in the sequence of the acoustic line signal frame data ds1 and one frame of acoustic line signal frame data ds0, which is a reference, (hereinafter the “reference acoustic line signal frame data ds0”) from the data storage 115 via the controller 116. The reference acoustic line signal frame data ds0 is a reference signal for extracting displacement due to a shear wave in the acoustic line signal frame data ds1 corresponding to each transmission event, and more specifically is acoustic line signal frame data acquired from the detection wave radiation region Ax prior to push wave pulse ppp transmission. From a difference between the acoustic line signal frame data ds1 and the reference acoustic line signal frame data ds0, the displacement detector 109 detects displacement (image information movement) Ptij of the observation point Pij in the detection wave radiation region Ax of the acoustic line signal frame data ds1, and associates the displacement Ptij with the observation point Pij coordinates to generate displacement frame data pt1 (1 being a natural number from 1 to m; the displacement frame data pt1 when numbers are not distinguished). The displacement detector 109 outputs the generated displacement frame data pt1 to the data storage 115.

7. Propagation Information Analyzer 110

The propagation information analyzer 110 is a circuit that determines an analysis target region from a plurality of observation points Pij in the region of interest roi on the basis of the time change characteristic of the displacement, calculates displacement peak frame data swf with respect to the analysis target region of the region of interest roi, and calculates propagation velocity frame data vo. The propagation information analyzer 110 includes an analysis target determiner 1101, a displacement peak extractor 1102, and a propagation velocity converter 1103.

(1) Analysis Target Determiner 1101

The analysis target determiner 1101 determines an analysis target region, which is a target of the propagation information analysis, on the basis of the time change characteristic of the displacement Ptij with respect to the observation points Pij present on the x coordinate with respect to each x coordinate of the region of interest roi. In the present embodiment, the analysis target region is information indicating a combination of coordinates i and j of each of the observation points Pij, which are targets of propagation velocity calculation.

Specifically, the analysis target determiner 1101 obtains displacement frame data pt1 from the data storage 115. The analysis target determiner 1101 obtains parameters indicating the time change characteristic of displacement, more specifically, sharpness of the peak of displacement, near the time at which the displacement data ptij is maximum with respect to each observation point Pij. The parameter indicating the sharpness of displacement peak is, for example, when the displacement data ptij is assessed as a function of time, a variance after approximation by Gaussian function, a half width at half maximum or full width at half maximum of the peak, continuous time in which the size of the displacement data ptij is a predetermined threshold or more, or the like. However, the parameter indicating the sharpness of the displacement peak is not limited to the above, but may be any parameter insofar as it is a parameter that indicates steepness of time change of displacement. Furthermore, the analysis target determiner 1101 specifies the observation point Pij at which the sharpness of the peak of the displacement is maximum with respect to each i and determines a combination of the specified i and j as an observation point included in the analysis target region.

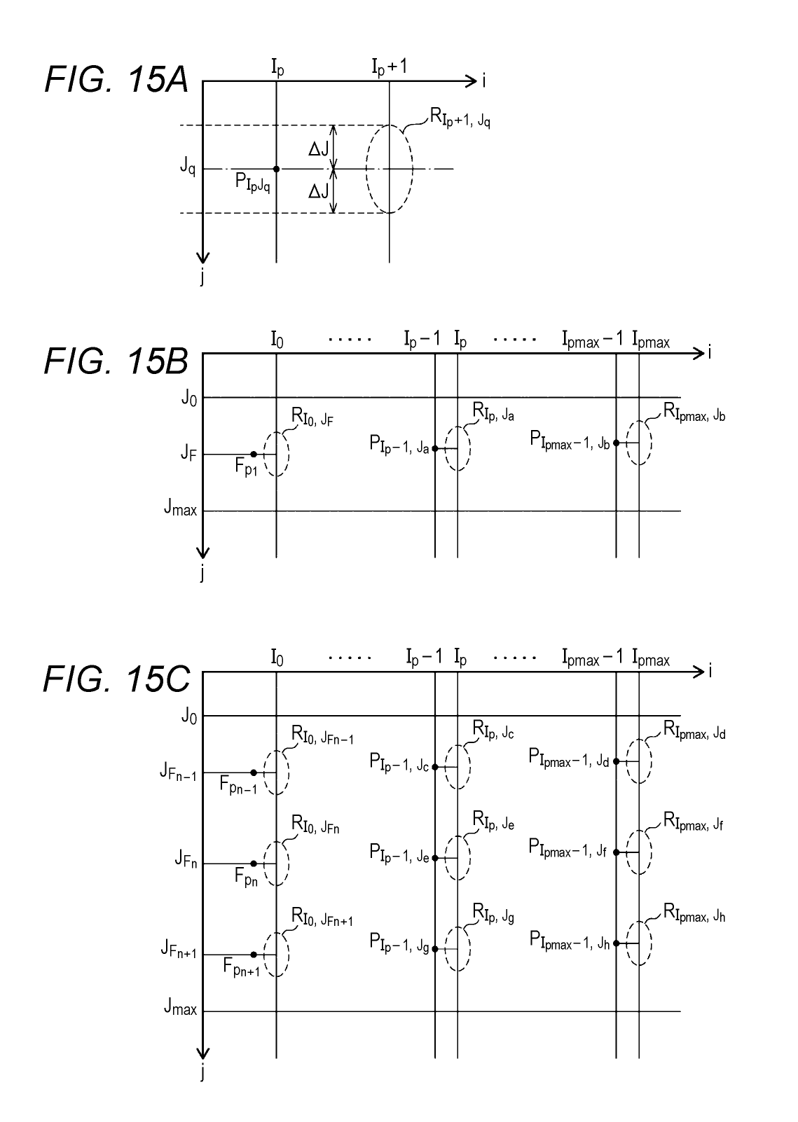

Specifically, the following operation is repeated: the observation point Pij, which is a reference in the region of interest roi, is specified, a region R, which is a search target, is set on an observation line adjacent in the i direction with reference to the position of the specified observation point Pij, and the observation point Pij at which the sharpness of the peak of the displacement is maximum in the region R is specified. FIG. 15A is a schematic diagram illustrating the operation in which the region R, which is a search target, is set on an observation line adjacent in the i direction with reference to the observation point Pij, which is a reference. In the description below, the observation point at which the i coordinate is I and the j coordinate is J is expressed as the observation point P_{IJ}. However, for the sake of clarity of I and J values, it may herein be expressed as the observation point P(I,J). Specifically, with reference to the observation point P(Iₚ,J_{q}) of i=I, at i=I+1 far from the transmission focus point FPn of the push wave, a range R(I_{p+q},J_{q}) of i=I+1, J_{q}−ΔJ≤j≤J_{q}+ΔJ in which width is 2×ΔJ is set with reference to the j coordinate J_{q} of the observation point P(Iₚ,J_{q}), and the observation point Pij at which the sharpness of the displacement peak is maximum is specified with respect to the region R(Iₚ+q,J_{q}). Note that the region R(I,J) is abbreviated as R_{I,J} in the drawings.

The case where the transmission focus point FPₙ of the push wave is Fp₁ only is described with reference to FIG. 15B. FIG. 15B illustrates the case where the range of the region of interest roi is I₀≤i≤Iₚₘₐₓ, J₀≤j≤Jₘₐₓ and the i coordinate of the transmission focus point Fp₁ is smaller than I₀. The analysis target determiner 1101 first sets a region R(I₀,j) with reference to the position of the transmission focus point Fp₁ instead of the specified observation point Pij with regard to an observation line of i=I₀ that is nearest the transmission focus point Fp₁. Specifically, with reference to the j coordinate J_{F} of the transmission focus point Fp₁, a region R(I₀,J_{F}) centering on the coordinate (I₀,J_{F}) is set. Then, the observation point Pij at which the sharpness of the displacement peak is maximum is specified with respect to the region R(I₀,J_{F}). Next, the analysis target determiner 1101 sets a region R(I₀+1,j) with reference to the position of the observation point Pij of specified i=I₀ with respect to the observation line of i=I₀+1 adjacent to the observation line of i=I₀ in the direction of moving away from the transmission focus point Fp₁, and specifies the observation point Pij at which the sharpness of the displacement peak is maximum with respect to the region R(I₀+1,j). Similarly, for example, the analysis target determiner 1101 sets a region R(Iₚ,Jₐ) on the observation line of i=Iₚ with reference to the position of the observation point P(Iₚ−1,Jₐ) of i=Iₚ−1 and specifies the observation point Pij at which the sharpness of the displacement peak is maximum with respect to the region R(Iₚ,Jₐ). Similarly, the analysis target determiner 1101 sets a region R(Iₚₘₐₓ,J_{b}) on the observation line of i=Iₚₘₐₓ with reference to the position of the observation point P(Iₚₘₐₓ−1,J_{b}) of i=Iₚₘₐₓ−1 and specifies the observation point Pij at which the sharpness of the displacement peak is maximum with respect to the region R(Iₚₘₐₓ,J_{b}). Thus, the observation point Pij is specified one by one with respect to every i of I₀≤I≤Iₚₘₐₓ.

In the case of the presence of a plurality of transmission focus points FPn of a push wave, the operation illustrated in the schematic diagram of FIG. 15C is performed. The analysis target determiner 1101 first specifies a region R for the number of transmission focus points Fpₙ with reference to the position of each transmission focus point Fpₙ in place of the specified observation point Pij with respect to the observation line of i=I₀ nearest the transmission focus point Fpₙ. Specifically, for example, the analysis target determiner 1101 sets a region R(I₀,J_{F(n-1)}) centering on a coordinate (I₀,J_{F(n-1)}) with reference to j coordinate J_{F(n-1)} of the transmission focus point Fpₙ₋₁. Similarly, the analysis target determiner 101 sets a region R(I₀,J_{Fn}) centering on a coordinate (I₀,J_{Fn}) with reference to j coordinate J_{Fn} of the transmission focus point Fpₙ and a region R(I₀,J_{F(n+1)}) centering on a coordinate (I₀,J_{F(n+1)}) with reference to j coordinate J_{F(n+1)} of the transmission focus point Fpₙ₊₁. The analysis target determiner 1101 specifies the observation point Pij at which the sharpness of the displacement peak is maximum with respect to each of the set regions R. Next, the analysis target determiner 1101 sets a region R with reference to the position of the specified observation point Pij of i=I₀ with respect to the observation line of i=I₀+1 nearest the transmission focus point Fp₁, and specifies the observation point Pij at which the sharpness of the displacement peak is maximum with respect to each region R. Similarly, for example, the analysis target determiner 1101 sets regions R(Iₚ,J_{c}), R(Iₚ,Jₑ), and R(Iₚ,J_{g}) on the observation line of i=Iₚ with reference to the position of each of the observation points P(Iₚ−1,J_{c}), P(Iₚ−1,Jₑ), and P(Iₚ−1,J_{g}) of i=Iₚ−1. Then, the analysis target determiner 1101 specifies the observation point Pij at which the sharpness of the displacement peak is maximum with respect to each of the regions R(Iₚ,J_{c}), R(Iₚ,Jₑ), and R(Iₚ,J_{g}). Similarly, the analysis target determiner 101 sets regions R(Iₚₘₐₓ,J_{d}), R(Iₚₘₐₓ,J_{f}), R(Iₚₘₐₓ,Jₕ) on the observation line of i=Iₚₘₐₓ with reference to each position of the observation points P(Iₚₘₐₓ−1,J_{d}), P(Iₚₘₐₓ−1,J_{f}), and P(Iₚₘₐₓ−1,Jₕ) of i=Iₚₘₐₓ−1. Then, the analysis target determiner 1101 specifies the observation point Pij at which the sharpness of the displacement peak is maximum with respect to each of the regions R(Iₚₘₐₓ,J_{d}), R(Iₚₘₐₓ,J_{f}), R(Iₚₘₐₓ,Jₕ). Thus, the observation point Pij is specified for the number of transmission focus points Fpₙ with respect to every i of I₀≤i≤Iₚₘₐₓ.

In the aforementioned example, description is given of the case where the transmission focus point Fpₙ is outside the region of interest roi. However, the transmission focus point Fpₙ may be present in the region of interest roi. In this case, when the coordinate of the transmission focus point Fpₙ is (I_{fn},J_{fn}), a region R (I_{fn-1},J_{fn}) centering on a coordinate (I_{fn}−1,J_{fn}) on the observation line of i=I_{fn}−1 and a region R (I_{fn+1},J_{fn}) centering on a coordinate (I_{fn+1},J_{fn}) on the observation line of i=I_{fn}+1 are set, and the observation points Pij are specified. Setting of the region R and specification of the observation point Pij is repeatedly performed in both directions of a direction in which i increases with distance from i=I_{fn} and a direction in which i decreases.

(2) Displacement Peak Extractor 1102

The displacement peak extractor 1102 specifies time at which the displacement data ptij is maximum with respect to each observation point Pij present in the analysis target region of the region of interest roi, generates displacement peak frame data swf associated with the position of the wavefront of the time at as the observation point Pij and outputs it to the data storage 115.

(3) Propagation Velocity Converter 1103

The propagation velocity converter 1103 converts the displacement peak frame data swf into propagation velocity data vij at the observation point Pij present in the analysis target region of the region of interest roi, generates propagation velocity frame data vo and outputs it to the data storage 115.

8. Elastic Modulus Calculator 111

The elastic modulus calculator 111 is a circuit that calculates the elastic modulus of a tissue with respect to the observation point Pij in the region of interest roi and calculates elasticity modulus frame data elf with respect to the region of interest roi. The elastic modulus calculator 111 includes an elastic modulus converter 1111. The elastic modulus converter 1111 treats propagation velocity data vo as an input, converts propagation velocity data v into elastic modulus data el at the observation point Pij in the region of interest roi, generates elasticity modulus frame data elf with respect to the region of interest roi, and outputs it to the data storage 115.

9. Other Configuration

The data storage 115 is a recording medium that sequentially records generated reception signal array rf, acoustic line signal frame data ds1 sequence, displacement frame data pt1 sequence, displacement peak frame data swf, propagation velocity frame data v1, and elastic modulus frame data e1.

The controller 116 controls each block in the ultrasound diagnostic apparatus 100 on the basis of an instruction from the operation inputter 102. As the controller 116, a processor such as a CPU can be used.

Further, although not illustrated, the ultrasound diagnostic apparatus 100 has a B mode image generator that generates ultrasound images (B mode images) in a time series based on components reflected from the tissue of the subject among acoustic line signals outputted based on ultrasound transmission and reception performed by the transmitter 106 and the detection wave receiver 108 without transmission of the push wave pulse ppp. The B mode image generator inputs acoustic line signal frame data from the data storage 115, performs processing such as envelope detection and logarithmic compression on the acoustic line signal to convert it to a luminance signal corresponding to the intensity, then subjects the luminance signal to coordinate transformation to an orthogonal coordinate system to generate B mode image frame data. Note that for ultrasound transmission and reception by the transmitter 106 and the detection wave receiver 108 for acquiring an acoustic line signal for B mode image generation, a publicly known method can be used. The generated B mode image frame data is outputted to the data storage 115 and stored therein. The display controller 113 configures a B mode image as a display image and causes the display 114 to display the display image.

Further, the elastic modulus calculator 111 may be configured to generate and display an elasticity image mapped to color information based on the elastic modulus indicated by the elastic modulus frame data elf. For example, an elasticity image may be generated in different colors in which coordinates at which elastic modulus is equal to or greater than a certain value are red, coordinates at which elastic modulus is less than the certain value are green, and coordinates at which elastic modulus could not be acquired are black. The operator's convenience can increase. The elastic modulus calculator 111 outputs the generated elastic modulus frame data elf and elasticity image to the data storage 115, and the controller 116 outputs the elasticity image to the display controller 113. Further, the display controller 113 may be configured to perform a geometric transformation on the elasticity image to transform it to image data for display, and output the geometrically transformed elasticity image to the display 114.

<Operation of Ultrasound Diagnostic Apparatus 100>

The operation of the integral SWS sequence of the ultrasound diagnostic apparatus 100 configured as described above is described.

1. Operation Outline

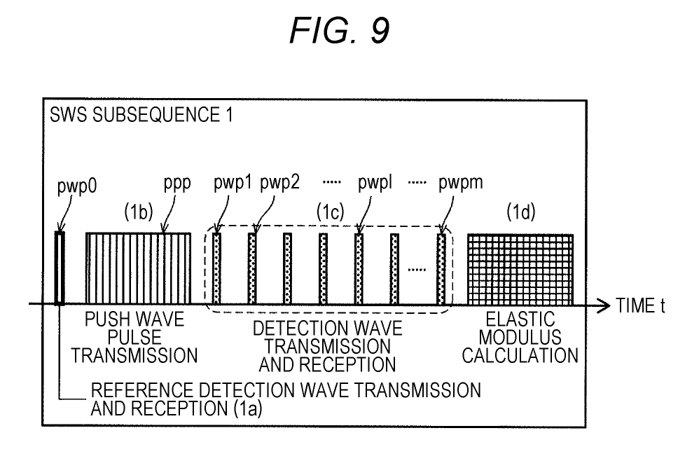

FIG. 9 is a schematic diagram illustrating an outline of an integral SWS sequence process in the ultrasound diagnostic apparatus 100. The SWS sequence by the ultrasound diagnostic apparatus 100 includes: a process in which reference detection wave transmission and reception is performed to obtain the reference acoustic line signal frame data ds0 for extracting displacement by a shear wave corresponding to each subsequent transmission event (1a), a process in which a push wave pulse pppₙ (n=1 to nₘₐₓ) is transmitted one or more times (nₘₐₓ times) to transmit a push wave ppₙ that converges to a specific site FP in the subject one or more times (nₘₐₓ times) to excite the shear wave in the subject (1b), a detection wave pulse pwp1 transmission and reception process in which transmission and reception of the detection wave pwp1 that passes by the region of interest roi is repeated multiple (m) times (1c), and an elastic modulus calculation process in which the shear wave propagation analysis is performed to calculate the shear wave propagation velocity of and the elastic modulus elf (1d).

2. SWS Sequence Operations

An operation of the ultrasound elastic modulus measurement processing after a B mode image is displayed on the display 114, in which the tissue is drawn based on reflection components from the tissue of the subject based on a publicly known method is described below.

Note that the B mode image frame data is generated such that, without transmission of the push wave pulse ppp, acoustic line signal frame data is generated in a time series based on reflected components from the tissue of the subject based on transmission and reception of ultrasounds by the transmitter 106 and the detection wave receiver 108, the acoustic line signal is then subjected to processing such as envelope detection and logarithmic compression so as to be converted into a luminance signal, and the luminance signal is then subjected to coordinate transformation to an orthogonal coordinate system. The display controller 113 causes the display 114 to display a B mode image in which the tissue of the subject is drawn.

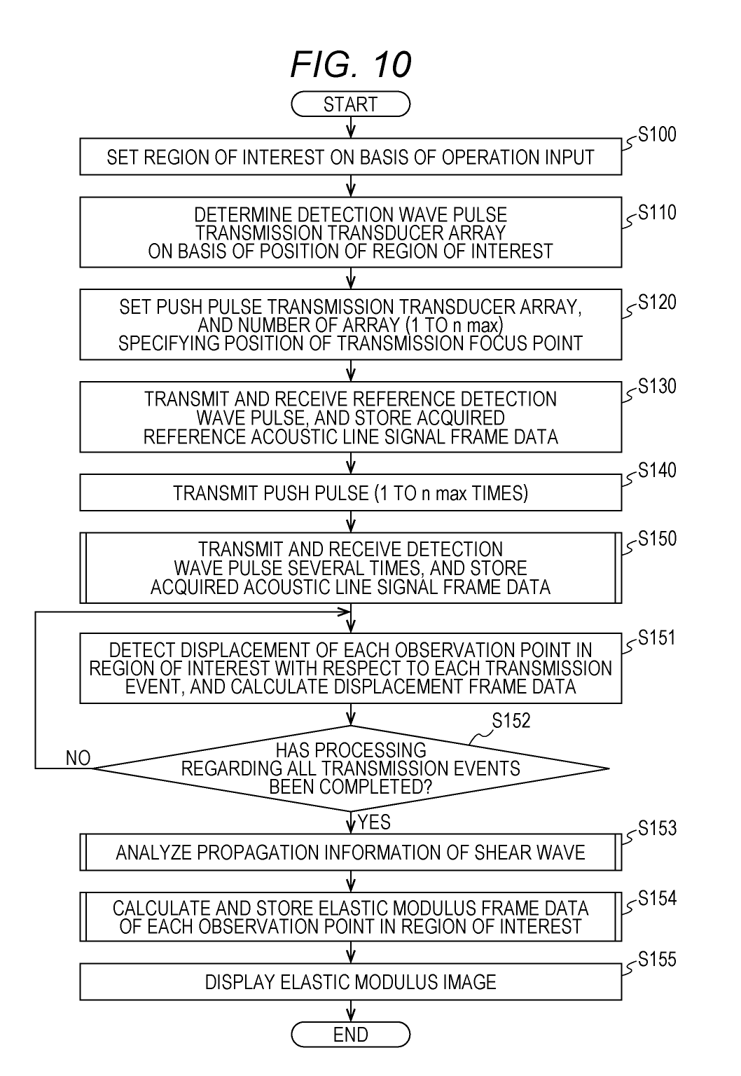

FIG. 10 is a flowchart illustrating an operation of ultrasound elastic modulus calculation of the ultrasound diagnostic apparatus 100.

[Steps S100 to S140]

In step S100, in a state in which a B mode image, which is a tomographic image of the subject acquired in real time by the probe 101, is displayed on the display 114, the region of interest setter 103 treats the information designated by the operator via the operation inputter 102 as an input and sets the region of interest roi indicating an analysis target range in the subject with reference to the position of the probe 101, and outputs the region of interest roi to the controller 116.

Designation of the region of interest roi by the operator is performed, for example, by displaying, on the display 114, the latest B mode image recorded on the data storage 115, and designating the region of interest roi via an inputter (not illustrated) such as a touch panel or a mouse. The region of interest roi may be, for example, an entire region of the B mode image, or a certain range including a middle portion of the B mode image.

In step S120, the push wave pulse generator 104 inputs the information indicating the region of interest roi through the controller 116 and sets the position of the transmission focus point FPn of the push wave pulse pppₙ (n=1 to nₘₐₓ) and the push wave transmission transducer array Pxₙ. In this example, as illustrated in FIG. 3A, the push wave transmission transducer array Pxₙ is constant regardless of the transmission order n of the push wave and includes all of the plurality of transducers 101a. In addition, the array direction transmission focus point position fx matches the array direction central position we of the detection wave radiation region Ax, and the depth direction transmission focus point position fyₙ (n=1 to nₘₐₓ) is present in the region of interest roi. However, the positional relationship between the detection wave radiation region Ax and the transmission focus point FP is not limited to the above, but may be changed as appropriate depending on the form or the like of a portion of the subject to be examined.

The information indicating the position of transmission focus point FP and the push wave transmission transducer array Px is outputted to the transmitter 106 as a transmission control signal together with the pulse width PWₙ of the push wave pulse ppp and the application start time PTₙ.

In step S130, the transmitter 106 transmits a detection wave pulse pwp0 to the transducer included in the detection wave transmission transducer array Tx to transmit a detection wave pw0 to the subject, and the detection wave receiver 108 receives reflected waves ec of the detection wave pw0 and generates the reference acoustic line signal frame data ds0, which is a reference for the tissue displacement. The reference acoustic line signal frame data ds0 is outputted to the data storage 115 and stored therein. A method of generating the acoustic line signal frame data is described later.

In step S140, the transmitter 106 causes the transducer included in the push wave transmission transducer array Pxₙ to transmit the push wave pulse pppₙ at least once (nₘₐₓ times) to cause the transducer to transmit the push wave ppn at least once (nₘₐₓ times) that converges an ultrasound beam to a specific site in the subject corresponding to the transmission focus point FP.

More specifically, the transmitter 106 generates a transmission profile based on the transmission control signal including the information indicating the position of the transmission focus point FPₙ and the push wave transmission transducer array Pxₙ acquired by the push wave pulse generator 104, the pulse width PWₙ of the push wave pulse pppₙ, and the application start time PTₙ. The transmission profile includes a pulse signal sp and delay time tpk with respect to each transmission transducer included in the push wave transmission transducer array Pxₙ. Then, the push wave pulse pppₙ is supplied to each transmission transducer based on the transmission profile. Each transmission transducer transmits the pulsed push wave ppₙ that converges to the specific site in the subject. The transmitter 106 performs this operation at least once (nₘₐₓ times).

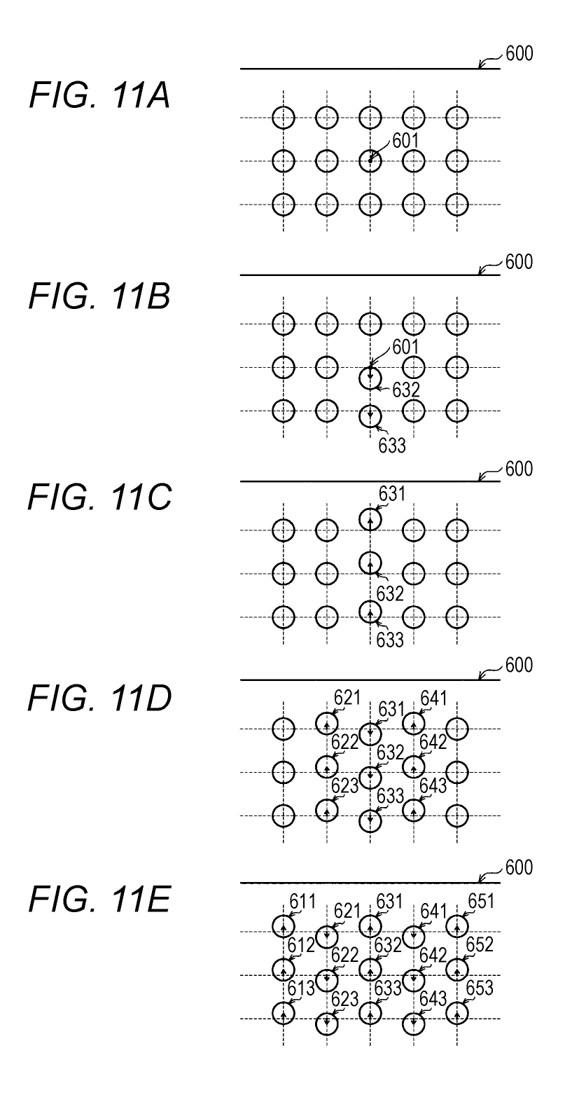

Here, the generation of the shear wave by the push wave pp is described with reference to the schematic diagrams of FIGS. 11A to 11E. FIGS. 11A to 11E are schematic diagrams illustrating the state of the generation of the shear wave by the push wave pp. FIG. 11A is a schematic diagram illustrating the tissue prior to the application of the push wave pp in a region in the subject corresponding to the detection wave radiation region Ax. In FIGS. 11A to 11E, each circle indicates a portion of the tissue in the subject and intersections of the dashed lines indicate the center positions of the circled tissues under the absence of load.

Here, when the push wave pp is applied to a focus point 601 in the subject corresponding to the transmission focus point FP with the probe 101 being in close contact with a skin surface 600, a tissue 632 positioned in the focus point 601 is pushed and moved in the traveling direction of the push wave pp, as illustrated in the schematic diagram of FIG. 11B. Further, a tissue 633, which is located on the side of the travel direction of the push wave pp from the tissue 632, is pushed by the tissue 632 and moved in the traveling direction of the push wave pp.

Next, when the transmission of the push wave pp ends, the tissues 632 and 633 tend to return to the original positions, and therefore the tissues 631 to 633 start vibrating along the traveling direction of the push wave pp as illustrated in the schematic diagram of FIG. 11C.

As illustrated in the schematic diagram of FIG. 11D, vibrations propagate to tissues 621 to 623 and tissues 641 to 643, which are adjacent to the tissues 631 to 633.

Further, as illustrated in the schematic diagram of FIG. 11E, the vibrations further propagate to tissues 611 to 663 and tissues 651 to 653. Accordingly, in the subject, the vibrations propagate in a direction perpendicular to the direction of the vibrations. In other words, the shear wave is generated at a point of the application of the push wave pp, and propagates in the subject.

[Step S150]

Description continues with reference back to FIG. 10.

In step S150, the detection wave pulse pwp1 is transmitted and received multiple times with respect to the region of interest roi, and the acquired acoustic line signal frame data ds1 sequence is stored. More specifically, the transmitter 106 causes the transducer included in the detection wave transmission transducer array Tx to transmit the detection wave pulse pwp1 to the subject, and the detection wave receiver 108 generates the acoustic line signal frame data ds1 based on the reflected waves ec received by the transducer included in the detection wave pulse reception transducer array Rx. Immediately after the end of transmission of the last push wave ppₙₘₐₓ, the above processing is repeated, for example, 10,000 times per second. Thus, immediately after the shear wave generation and until the propagation ends, the acoustic line signal frame data ds1 in the detection wave radiation region Ax of the subject is repeatedly generated. The generated acoustic line signal frame data ds1 sequence is outputted to the data storage 115 and stored therein.

Step S150 is described in detail below.

First, regarding an arbitrary observation point Pij present in the detection wave radiation region Ax, the detection wave receiver 108 calculates the transmission time taken for the transmitted ultrasound reaches the observation point Pij in the subject. The transmission time is calculated when the shortest path from the detection wave transmission transducer array Tx to the observation point Pij is divided by the speed of sound cs of the ultrasound.

Next, the detection wave receiver 108 sets the detection wave pulse reception transducer array Rx, and calculates the reception time taken for the reflected detection wave from the observation point Pij reaches the reception transducers Rwk included in the detection wave pulse reception transducer array Rx. The reception time is calculated when the shortest path from the observation point Pij to the reception transducer Rwk is divided by the speed of sound cs of the ultrasound.

Then, the detection wave receiver 108 calculates a delay from the transmission time and the reception time with respect to each observation point Pij and with respect to each reception transducer Rwk, and identifies reception signals from the observation points Pij with respect to each observation point Pij from the acoustic line signal frame data ds1.

Next, the detection wave receiver 108 performs weighted summing of the reception signals identified with respect to each observation point Pij and calculates an acoustic line signal with respect to the observation point Pij. Here, for weighting, reception apodization is performed such that weighting is maximum with respect to the transducer centrally positioned in the x direction of the detection wave pulse reception transducer array Rx.

The detection wave receiver 108 stores the calculated acoustic line signal in the data storage 115.

[Step S151]

In step S151, the displacement detector 109 detects the displacement of the observation point Pij in the region of interest roi for each transmission event.

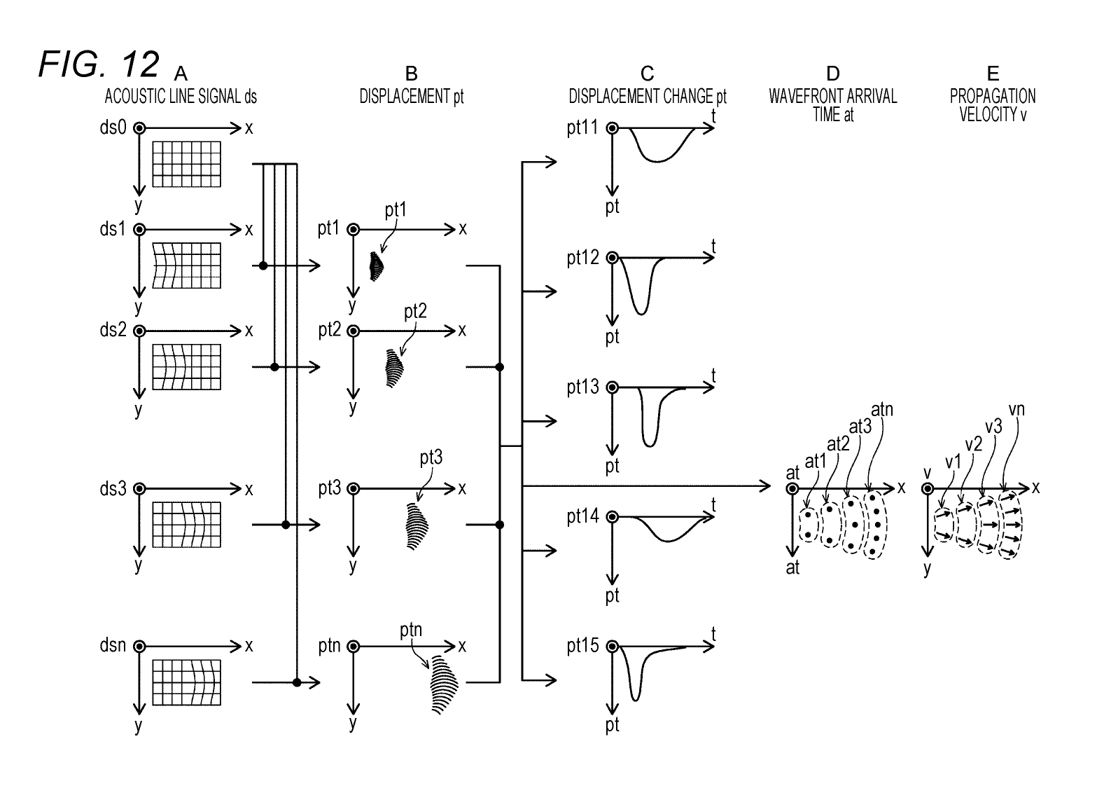

FIG. 12 is a schematic diagram illustrating displacement detection and a shear wave propagation analysis operation.

First, the displacement detector 109 acquires the reference acoustic line signal frame data ds0 stored in the data storage 115 in step S130. As described above, the reference acoustic line signal frame data ds0 is acoustic line signal frame data acquired prior to the transmission of the push wave pp, i.e., prior to the generation of the shear wave.

Next, the displacement detector 109 detects displacement of each pixel at the time of the acquisition of the acoustic line signal frame data ds1 from a difference between the acoustic line signal frame data ds1 and the reference acoustic line signal frame data ds0 with respect to each acoustic line signal frame data ds1 stored in the data storage 115 in step S150.

In FIG. 12, the array A indicates the reference acoustic line signal frame data ds0 and the acoustic line signal frame data ds1 generated at each transmission event, and the array B indicates the displacement frame data pt1 calculated with respect to each transmission event in step S150. As indicated by the array A and the array B of FIG. 12, the displacement frame data pt1 is detected in such a manner that the acoustic line signal frame data ds1 is compared with the reference acoustic line signal frame data ds0 to detect which acoustic line signal dsij′ of an observation point P′ij′ of the acoustic line signal frame data ds1 resembles the acoustic line signal dsij of the observation point Pij in the reference acoustic line signal frame data ds0, and positional displacement of the observation point P′ij′ with respect to the observation point Pij is calculated.

Specifically, for example, correlation processing is performed between the acoustic line signal frame data ds1 and the reference acoustic line signal frame data ds0 to specify the observation point P′ij′ corresponding to the observation point Pij and a distance j′−j between the observation points is specified as displacement of the observation point Pij.

Note that the method for specifying displacement is not limited to the correlation processing between two acoustic line signals that share the i coordinate, but may be pattern matching.

The displacement detector 109 generates displacement data ptij of the observation point in the region of interest roi by associating the displacement of each observation point Pij pertaining to one frame of acoustic line signal frame data ds1 with the coordinates ij of the observation point, and outputs the generated displacement frame data pt1 pertaining to the region of interest roi to the data storage 115.

[Steps S152 to S155]

The propagation information analyzer 110 outputs the generated displacement frame data pt1 to the data storage 115 and the generated displacement frame data pt1 is stored (step S151). Whether the processing of step S151 is completed for all specified transmission events is determined (step S152). If not completed, the processing returns to step S151 and a series of processing is performed for the transmission of a next detection wave pulse pwp1. If completed, the processing proceeds to step S153.

In step S153, the propagation information analyzer 110 determines an analysis target region on the basis of the time change characteristic of the displacement Ptij with respect to the observation points Pij in the region of interest roi. Next, the propagation information analyzer 110 detects the time when the displacement is maximum regarding the observation point present in the analysis target region, and generates the displacement peak frame data swf in which the position of the observation point Pij is associated with the time when the displacement is maximum. Furthermore, the propagation information analyzer 110 converts the displacement peak frame data swf into propagation velocity data vij at the observation point Pij present in the analysis target region of the region of interest roi, generates the propagation velocity frame data vo, and outputs it to the data storage 115. Details of the shear wave propagation information analysis method in step S153 will be described later.

In step S154, the elastic modulus calculator 111 calculates the elastic modulus data elij with respect to the observation point Pij in the region of interest roi, calculates the elasticity modulus frame data elf with respect to the region of interest roi, and outputs it to the data storage 115. Details of the method for calculating the elasticity modulus frame data elf in step S154 will be described later.

In step S155, the elastic modulus calculator 111 generates an elasticity image on which color information has been mapped on the basis of the elastic modulus indicated by the elasticity modulus frame data elf. Specifically, for example, an observation point at which the elastic modulus is a predetermined threshold or more is red, an observation point at which the elastic modulus is less than a predetermined threshold is green, and an observation point at which the elastic modulus is not calculated is black. Note that the color information mapping is not limited to the above example, but three or more colors may be applied depending on the elastic modulus, and an observation point at which the elastic modulus is not calculated may be grey or white. In addition, when an elasticity image is superimposed on a B mode tomographic image, the color to be superimposed on an observation point at which the elastic modulus is not calculated may be transparent (B mode tomographic image is left as it is). The display controller 113 performs a geometric transformation on the elasticity image into image data for screen display, and outputs the geometrically transformed elasticity image to the display 114.

Thus, the SWS sequence processing illustrated in FIG. 10 is completed. According to the ultrasound elastic modulus measurement processing above, the elastic modulus frame data elf by the SWS sequence can be calculated.

3. Details of Processing in Step S153

In step S153, the propagation information analyzer 110 determines an analysis target region on the basis of the time change characteristic of the displacement Ptij with respect to observation points Pij in the region of interest roi. Next, the propagation information analyzer 110 detects the time when the displacement is maximum regarding the observation point present in the analysis target region, and generates the displacement peak frame data swf in which the position of the observation point Pij is associated with the time when the displacement is maximum. Furthermore, the propagation information analyzer 110 converts the displacement peak frame data swf into propagation velocity data vij at the observation point Pij present in the analysis target region of the region of interest roi, generates the propagation velocity frame data vo, and outputs it to the data storage 115.

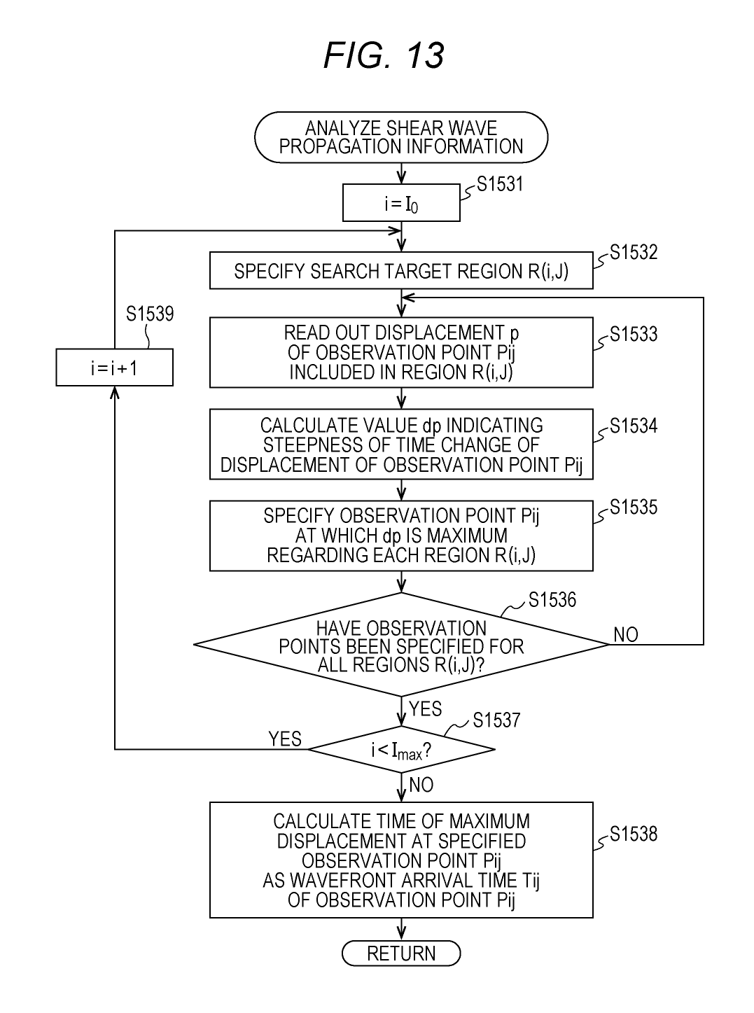

Details are described in conjunction with the flowchart of FIG. 13. FIG. 13 is a flowchart illustrating a shear wave propagation information analysis operation.

First, parameter i indicating the i coordinate of the observation point Pij is initialized (step S1531). Next, a search target region R(i,J) is specified (step S1532). As described above, for first i, the region R(i,J) is specified on the basis of the j coordinate of the push wave transmission focus point Fpₙ, and for the second and subsequent i, the region R(i,J) is specified on the basis of an observation point P(i−1,j) of specified i=i−1.

Next, the displacement p of the observation point Pij included in the region R(i,J) is read out (step S1533). Then, the parameter dp indicating a time change of the displacement p of the observation point Pij is calculated (step S1534).

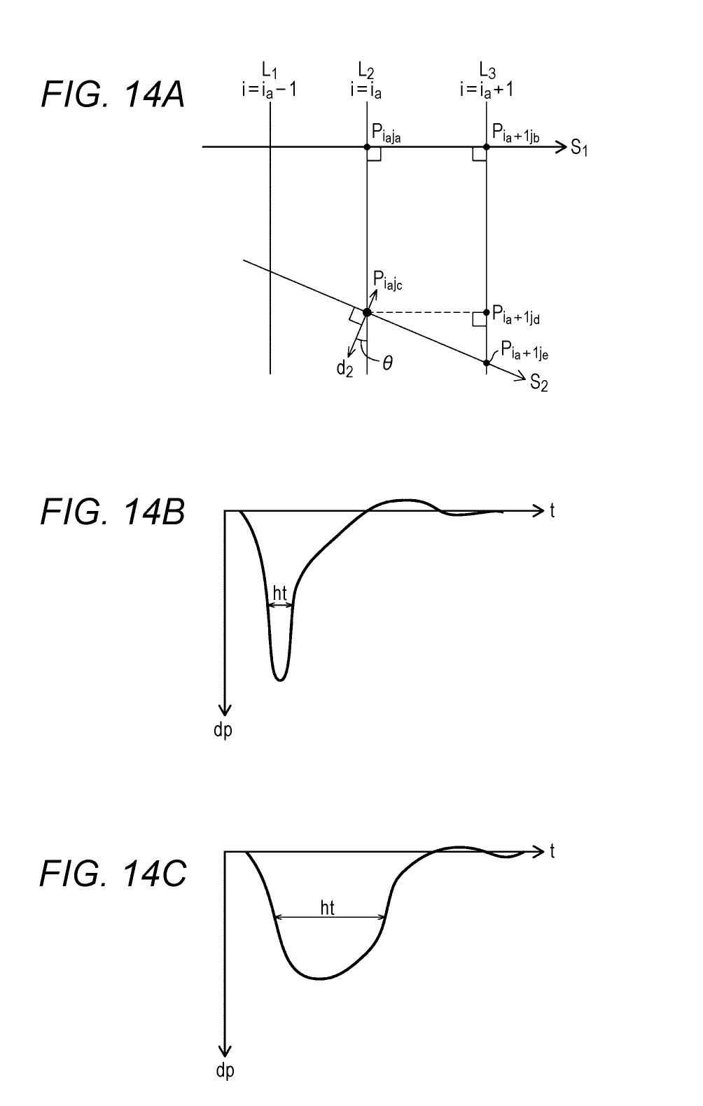

Description is given below with reference to the schematic diagrams of FIGS. 14A to 14C.

FIG. 14A schematically illustrates a relative relationship between observation lines L1, L2 and L3, observation points present thereon, and shear wave traveling directions. Herein, the observation point L1 is a straight region in which an observation point Pij with the i coordinate of i=iₐ−1 is present. Similarly, the observation point L2 is a straight region in which an observation point Pij with the i coordinate of i=iₐ is present, and the observation point L3 is a straight region in which an observation point Pij with the i coordinate of i=iₐ+1 is present. Here, the shear wave is a wave perpendicular to the traveling direction of the wave and the direction of wave vibration. Therefore, the displacement by the shear wave is maximum in a tangential direction of the wavefront. Therefore, at an observation point Piₐjₐ at which a shear wave traveling direction S1 and the observation line L2 are substantially perpendicular, the direction of the displacement by the shear wave substantially matches the direction of the observation point L2. Similarly, at an observation point Piₐ+1j_{b} at which the shear wave traveling direction S1 and the observation line L3 are substantially perpendicular, the direction of the displacement by the shear wave substantially matches the direction of the observation point L3. At this time, a line segment connecting the two points: the observation point Piₐjₐ the observation point Piₐ+1j_{b} substantially matches the shear wave path. Therefore, when the distance between the two points: the observation point Piₐjₐ and the observation point Piₐ+1j_{b} is divided by a time difference between the times at which the displacement is maximum with respect to the two points: the observation point Piₐjₐ and the observation point Piₐ+1j_{b}, the velocity of the shear wave between the two observation points can be calculated precisely.

Note that FIG. 14A illustrates the case where the observation lines L1, L2 and L3 are parallel to one another and arranged at equal intervals. However, the relationship of the observation lines L1, L2 and L3 is not limited to the above case. For example, the distance between the observation lines L1 and L2 may differ from the distance between the observation lines L2 and L3. In addition, for example, the depth indicated by the same j coordinate may not be the same between the observation lines L1, L2 and L3. In addition, the observation lines L1 to L3 may not be parallel. For example, the observation lines L1, L2 and L3 may be set in a radial fashion to intersect at a certain point. Furthermore, the observation lines L1, L2 and L3 may not be a straight line, but a curved line. Even in a case the shear wave does not propagate in the horizontal direction (x direction), when the observation line is set to be substantially perpendicular to the shear wave traveling direction, the velocity of the shear wave can be calculated precisely.

Thus, the analysis target determiner 1101 specifies the observation point Pij at which the shear wave traveling direction and the observation line are substantially perpendicular as described above, as an observation point included in the analysis target region. Specifically, the analysis target determiner 1101 calculates the parameter dp indicating the time change of the displacement p in a direction along the observation point of the observation point Pij. In the present embodiment, for the parameter dp, when the half width at half maximum of the peak when the displacement p is assessed as a function of time is ht[sec], an inverse number 1/ht of ht is used as the parameter dp. This is because the peak becomes sharper as the degree of matching between the shear wave propagation direction and the observation line direction increases. At the observation point Piₐjₐ at which the shear wave traveling direction S1 and the observation line L2 are perpendicular, the direction of the displacement by the shear wave matches the direction of the displacement p. Therefore, the displacement p has a large absolute value and provides a steep peak. Therefore, the time-series change of the displacement has a high peak and a sharp characteristic as indicated by the graph of FIG. 14B. The analysis target determiner 1101 specifies an observation point at which the shear wave traveling direction and the observation line are substantially perpendicular, like the observation point Piₐjₐ, as an observation point included in the analysis target region.

In contrast, for example, at the observation point Piₐj_{c} at which the shear wave traveling direction S1 and the observation line L2 are not substantially perpendicular, the displacement direction d2 by the shear wave and the direction of the observation line L2, i.e., the direction of the displacement p, form an angle θ. Therefore, the absolute value of the displacement p is smaller in proportion to the value of cos θ, and the peak is obtuse. In addition, the shear wave that passes by the observation point Piₐj_{c} passes by the observation point Piₐ+1 jₑ on the observation point L3. However, it cannot be specified whether the shear wave has passed by the observation point Piₐ+1 j_{d} or the observation point Piₐ+1 jₑ since the displacement p has an obtuse peak similarly at the observation point Piₐ+1 jₑ and at the observation point Piₐ+1 j_{d} which is the closest to the observation point Piₐjₑ. Therefore, it cannot be specified which observation point the shear wave that has passed by the observation point Piₐj_{c} passes by next, and thus the propagation distance of the shear wave cannot be specified precisely. Thus, the analysis target determiner 1101 does not specify the observation point at which the shear wave traveling direction and the observation line are not substantially perpendicular, like the observation point Piₐj_{c}, as an observation point included in the analysis target region.

The analysis target determiner 1101 calculates the parameter dp with respect to all the observation points included in the region R(i,J) (step S1534) and specifies the observation point Pij at which dp is maximum with respect to each of all the regions R(i,J) (steps S1535 and S1536). Then, i is incremented (S1539), the search target region R(i,J) is set on the basis of the coordinate of the observation point Pij specified in step S1535 (step S1532). The observation point Pij at which dp is maximum is specified with respect to each region R(i,J) (steps S1535 and S1536). The above operations are repeated (step S1537). Thus, the analysis target region is extracted from the entire region of interest roi.

Next, the propagation information analyzer 110 specifies the time at at which the displacement is maximum with respect to each observation point Pij included in the analysis target region and generates the displacement peak frame data swf as the wavefront arrival time Tij of the observation point Pij and outputs it to the data storage 115.

The array D of FIG. 12 is displacement peak frame data swf in which the time at which the displacement is maximum is plotted as a function value. The observation points circled by the dotted lines indicate observation points at which the wavefront arrival time is the same.

4. Details of Processing in Step S154

In step S154, the elastic modulus calculator 111 calculates the shear wave propagation velocity on the basis of the displacement peak frame data swf or the elastic modulus with respect to the observation point Pij included in the analysis target region in the region of interest roi, and calculates the elasticity modulus frame data elf.

First, the propagation velocity converter 1103 reads out the displacement peak frame data swf from the data storage 115 and converts it to the propagation velocity frame data vfo as described below. FIGS. 15A to 15C are schematic diagrams illustrating the method for calculating a wavefront propagation velocity. First, the propagation velocity converter 1103 specifies a shear wave propagation route by grouping the observation points included in the analysis target region specified by the analysis target determiner 1101 on the basis of the relationship between the region with the specified observation point and the observation point used as an index for the region. Specifically, in a case a region R(i+1,j) in which the i coordinate is i+1 is set on the basis of the position of the observation point Pij and an observation point P(i+1)j′ is specified from the region R(i+1,j), the observation point Pij is associated with the observation point P(i+1)j′. That is, the first observation point is associated with the second observation point that is specified from the region which is set on the basis of the position of the first observation point. A line connecting the associated observation points is a shear wave propagation route. Specifically, an observation point P1j₁ is associated with an observation point P2J₂ that is specified from the region which is set on the basis of the observation point P1j₁. Similarly, an observation point P2j₂ is associated with an observation point P3J₃ that is specified from the region which is set on the basis of the observation point P2j₂. Thus, as a shear wave propagation route, a polygonal line connecting observation points P1J₁-P2J₂-P3J₃-P4J₄-P5J₅-P6J₆ is specified. Similarly, as a shear wave propagation route, observation points P17J₇-P2J₈-P3J₉-P4J₁₀-P5J₁₁-P6J₁₂ are specified.

Then, the propagation velocity converter 1103 calculates a shear wave velocity by dividing the distance between the associated two observation points by a difference of the displacement peak times of the observation points. That is, vij={T(i+1)j′−Tij}/d

where Tij is the displacement peak time of the observation point Pij, T(i+1)j′ is the displacement peak time of the observation point P(i+1)j′, d is the distance between the observation point Pij and the observation point P(i+1)j′.

The elastic modulus converter 1111 converts the propagation velocity frame data vfo into the elasticity modulus frame data elf. The elastic modulus Eij of the observation point Pij can be calculated by the formula below.

Eij=K×vij²

where, K is a constant of approximately 3.

The array E of FIG. 12 is propagation velocity frame data of calculated from wavefront arrival time frame data a calculated with respect to each transmission event.

Thus, the calculated elastic modulus Eij is converted to color information indicating the elastic modulus Eij and the color information is mapped to the position of the corresponding observation point Pij such that the elasticity image can be formed.

In the aforementioned procedure, the elastic modulus calculator 111 generates the elasticity modulus frame data elf and stores it in the data storage 115 (step S1554).

Thus, the calculation processing for the elastic modulus measurement on the basis of the shear wave propagation analysis is completed.

<Summary>

With the aforementioned configuration, the propagation analysis is performed only at the observation point where the shear wave propagation direction is the closest to the perpendicular state with respect to the observation line. Therefore, the precision of the propagation analysis can be increased with respect to the shear wave that propagates to be substantially perpendicular to the observation line, i.e., the shear wave in which the observation line is substantially parallel to the wavefront. Furthermore, when observation lines and observation points are appropriately arranged, not in a grid-like mesh pattern, even when the shear wave propagation direction is any direction, the precision of the propagation analysis can be increased. Therefore, with the aforementioned configuration, it is possible to increase the precision of the propagation analysis.

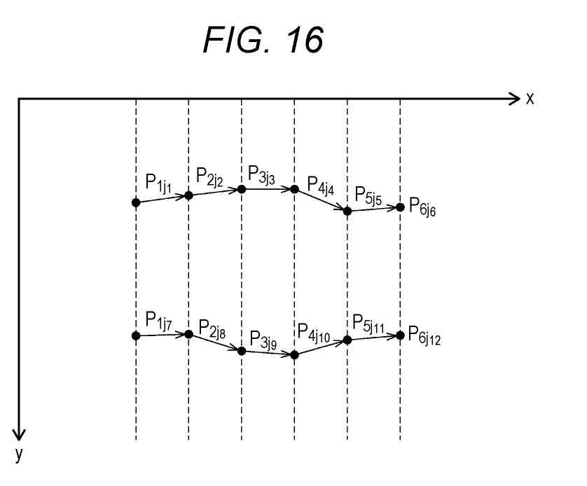

In addition, when an observation point Pij included in the analysis target region is specified with respect to one observation line and then the observation point Pij on an observation line adjacent on the side farther from the push wave transmission focus point FPn is specified, the search range may be limited to an area near the observation point Pij that has already been specified. As illustrated in FIG. 16, the shear wave that passes by the observation point Pij propagates in a direction substantially perpendicular to the observation line at the observation point Pij. Therefore, there is a high possibility that a different observation point exists on or near a straight line that passes by the observation point Pij and is perpendicular to the observation line at the observation point Pij. With such configuration, the amount of calculation can be reduced.

<<Variation>>

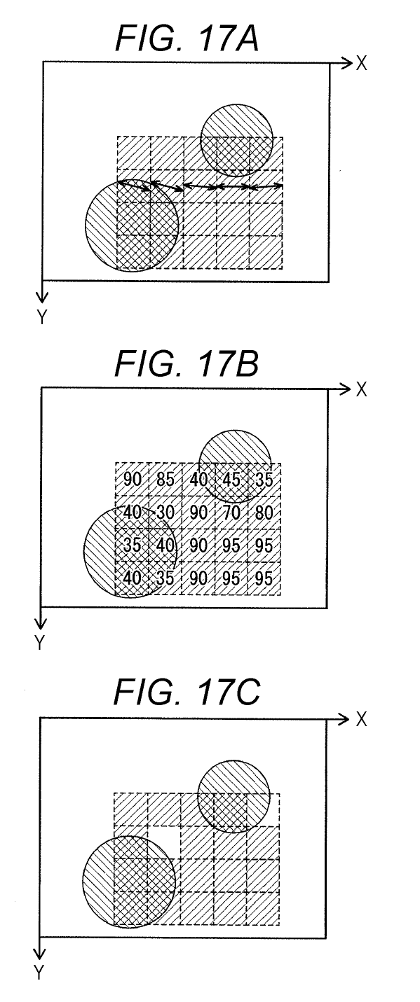

(1) In an embodiment, the target of the propagation analysis and the display of the results are limited to the observation point Pij present in the analysis target region, but may be performed as described below. For example, the detection of the time of the displacement peak, the calculation of the propagation velocity of the shear wave, and the conversion to the elastic modulus may be performed with respect to all the observation points in the region of interest, and then the information indicating the analysis target region or the parameter dp indicating the time change of the displacement p of the observation point Pij may be superimposed on the elasticity image. For example, as illustrated in an example of an enlarged elasticity image of FIG. 17A, an arrow indicating an analysis target region may be displayed over the elasticity image. Alternatively, for example, as illustrated in an example of an enlarged elasticity image of FIG. 17B, a parameter dp indicating the time change of the displacement p of each observation point may be displayed over the elasticity image. In the example of FIG. 17B, inverse number 1/ht of half width at half maximum ht of the peak when the displacement is assessed as a function of time is standardized such that the maximum number does not exceed 100, and values rounded with increments of 5 are displayed. Alternatively, as in the case of an example of an enlarged elasticity image of FIG. 17C, the detection of the time of the displacement peak, the calculation of the propagation velocity of the shear wave, and the conversion to the elastic modulus may also be performed on an observation point not included in the analysis target region, and then the color mapping may not be performed for an observation point at which the parameter dp does not meet a predetermined reference.

(2) In the embodiment, as the parameter dp indicating the time change of the displacement p of the observation point, an inverse number 1/ht of half width at half maximum ht of the peak when the displacement is assessed as a function of time is used. However, as described above, any value indicating sharpness (steepness) of peak of the time change of the displacement p of the observation point may be used. For example, an inverse number of full width at half maximum of the peak when the displacement is assessed as a function of time, a variance after approximation by Gaussian function, or the degree of matching with the reference peak may be used.

(3) In the embodiment, the ultrasound diagnostic apparatus 100, prior to the process of push wave pulse transmission, performs the process of reference detection wave pulse transmission and reception, and the displacement detector detects displacement Ptij of the observation point Pij on the basis of the difference between the acoustic line signal frame data ds1 and the reference acoustic line signal frame data ds0 formed by the reference detection wave pulse transmission and reception, and generates the displacement frame data pt1 by associating the displacement Ptij with the coordinate of the observation point Pij. However, the method for detecting the tissue displacement is not limited to the above case. For example, the ultrasound diagnostic apparatus does not perform the process of reference detection wave pulse transmission and reception and does not generate the reference acoustic line signal frame data ds0. Then, the displacement detector detects change ΔPtij of the displacement Ptij of the observation point Pij between transmission events on the basis of a difference between the acoustic line signal frame data ds1 and the acoustic line frame data ds(1−1) obtained at the last transmission event. Then, the change ΔPtij of the displacement Ptij between transmission events is accumulated with respect to each observation point Pij to generate displacement Ptij of the observation point Pij. Then, the displacement Ptij may be associated with the coordinate of the observation point Pij to generate the displacement frame data pt1. Note that the detection of the change ΔPtij between transmission events is not limited to between two continuous transmission events, but the change ΔPtij of the displacement Ptij of the observation point Pij may be calculated from a difference between any two acoustic line signal frame data ds1.

(4) For the ultrasound diagnostic apparatuses according to the embodiment and the variations, all or part of their constituent elements may be achieved by one chip or an integrated circuit of chips, or a computer program, or carried out in any other form. For example, the propagation analyzer and the assessor may be achieved by one chip, the ultrasound signal acquirer only may be achieved by one chip, and the displacement detector or the like may be achieved by a different chip.

When it is achieved by an integrated circuit, typically it is achieved as a Large Scale Integration (LSI). Herein, an LSI is used. However, it may be called an IC, a system LSI, a super LSI, or an ultra LSI depending on difference in degree of integration.

In addition, the manner of an integrated circuit is not limited to an LSI, but may be achieved by a dedicated circuit or a general-purpose processor. After the LSI is manufactured, a Field Programmable Gate Array (FPGA), which is programmable, or a reconfigurable processor that is reconfigurable for connection or setting of a circuit cell in an LSI may be used.

Furthermore, when an integrated circuit technology that replaces the LSI turns into reality because of progress of semiconductor technology or by a derived, different technology, of course, integration of a functional block may be performed using such technology.

In addition, the ultrasound diagnostic apparatuses according to the embodiments and variations may be achieved by a program written in a recording medium and a computer that reads and executes the program. The recording medium may be any recording medium such as a memory card or a CD-ROM. In addition, the ultrasound diagnostic apparatus according to the embodiment of the present invention may be achieved by a program downloaded via a network and a computer that downloads a program from a network and executes the program.

(5) The embodiments described above indicate preferable specific examples of the present invention. The values, the shapes, the materials, the constituent elements, the arrangement positions and connection forms of the constituent elements, the processes, and the order of the processes indicated in the embodiments are examples, but do not limit the present invention. In addition, among the constituent elements of the embodiments, the processes not stated in the independent claims indicating the most generic concept of the present invention are described as given constituent elements that constitute more preferable forms.

In addition, for the sake of easy understanding of the invention, the scale of the constituent elements in the drawings indicated in the embodiments described above may be different from the actual scale. In addition, the present invention is not limited to what is described in the embodiments described above, but may be appropriately changed without departing from the gist of the present invention.

Furthermore, the ultrasound diagnostic apparatus includes members including a circuit component and a lead wire on a substrate, and can be carried out in various aspects with respect to an electrical wiring and an electrical circuit on the basis of ordinary knowledge in the present technical field, but they have no direct relevance with the description of the present invention and therefore will not be elaborated. Note that the drawings indicated above are schematic diagrams, and are not necessarily illustrated strictly.

<<Supplement>>

(1) The ultrasound diagnostic apparatus according to the embodiment is an ultrasound diagnostic apparatus to which a probe including a plurality of transducers arranged can be connected, causing the probe to transmit a push wave in which ultrasound beams are converged into a subject to detect propagation velocity of a shear wave generated by acoustic radiation pressure of the push wave, and the ultrasound diagnostic apparatus includes: a push wave pulse transmitter that uses a plurality of transmission transducers selected from the plurality of transducers to transmit a push wave that converges to one or more transmission focus points in the subject; a detection wave pulse transmitter that supplies a detection wave pulse to some or all of the plurality of transducers to cause the plurality of transducers to transmit, following transmission of the push wave, a detection wave that passes by a region of interest indicating an analysis target range in the subject multiple times; a displacement detector that detects displacement of a tissue at each of a plurality of observation points in the region of interest on the basis of reflected detection waves received in a time series by the plurality of transducers corresponding to each of detection waves of the multiple times; an analysis target determiner that determines an analysis target region, that is a target of shear wave propagation analysis, on the basis of steepness of a time change of displacement of the tissue at the plurality of observation points; and a propagation information analyzer that calculates the propagation velocity of the shear wave at each observation point present in the analysis target region on the basis of displacement of the tissue at the plurality of observation points present in the analysis target region.

In addition, the ultrasound signal processing method according to the embodiment is an ultrasound signal processing method using a probe including a plurality of transducers arranged to transmit a push wave in which ultrasound beams are converged into a subject to detect propagation velocity of a shear wave generated by acoustic radiation pressure of the push wave, and the ultrasound signal processing method includes: by using a plurality of transmission transducers selected from the plurality of transducers, transmitting a push wave that converges to one or more transmission focus points in the subject; by supplying a detection wave pulse to some or all of the plurality of transducers, causing the plurality of transducers to transmit, following transmission of the push wave, a detection wave that passes by a region of interest indicating an analysis target range in the subject multiple times; detecting displacement of a tissue at each of a plurality of observation points in the region of interest on the basis of reflected detection waves received in a time series by the plurality of transducers corresponding to each of detection waves of the multiple times; determining an analysis target region, that is a target of shear wave propagation analysis, on the basis of steepness of a time change of displacement of the tissue at the plurality of observation points; and calculating the propagation velocity of the shear wave at each observation point present in the analysis target region on the basis of displacement of the tissue at the plurality of observation points present in the analysis target region.

According to the present disclosure, with the aforementioned configuration, propagation analysis of shear wave is performed with regard to a region in which the propagation direction of the shear wave is the same as the supposed direction in the subject, and therefore it is possible to suppress mismatching due to misalignment of the propagation direction of the shear wave to increase the propagation analysis precision. In addition, because it is not necessary to analyze the propagation direction of the shear wave, it is possible to reduce the amount of calculation for propagation analysis.

(2) In addition, in the ultrasound diagnostic apparatus according to (1) above, the propagation information analyzer may specify a time when a value of displacement is maximum with regard to each observation point present in the analysis target region and treat the specified time as a time when the shear wave passed by the observation point to calculate velocity of the shear wave.

Thus, because the wavefront of the shear wave can be specified on the basis of the time when the value of the displacement is maximum, it is possible to perform the propagation analysis with the processing with less amount of calculation.

(3) In addition, in the ultrasound diagnostic apparatus according to (1) or (2) above, the analysis target determiner, on the basis of the time change of the displacement of the tissue at an observation point present at a depth of a predetermined range including a depth at which the transmission focus point is present, may select the analysis target region from the depth of the predetermined range.

Thus, it is possible to perform determination of the analysis target region only with regard to a region where the possibility that the shear wave propagating from the transmission focus point passes in a direction substantially perpendicular to the observation line is sufficiently high. Therefore, it is not necessary to determine the analysis target region across the entire region of the region of interest, and it is possible to reduce the amount of calculation.

(4) In addition, in the ultrasound diagnostic apparatus according to (1) or (2) above, the analysis target determiner, on the basis of the time change of the displacement of the tissue at an observation point present at a depth of a predetermined range including the analysis target region determined on an acoustic line adjacent on a side near the transmission focus point, may select an analysis target region on the acoustic line from the depth of the predetermined region.

Thus, it is possible to search the observation point included in the analysis target region by following the movement of the shear wave with regard to a region where the shear wave that has passed by the specified observation point can reach. Therefore, it is not necessary to determine the analysis target region across the entire region of the region of interest, and it is possible to reduce the amount of calculation.

(5) In addition, in the ultrasound diagnostic apparatus according to (1) to (4) above, the analysis target determiner may determine that a depth at which an observation point at which the time change of the displacement of the tissue is maximum presents among a plurality of observation points having different depth, as the analysis target region.

(6) In addition, in the ultrasound diagnostic apparatus according to (1) to (4) above, the analysis target determiner may determine that a depth at which an observation point at which a profile of the time change of the displacement of the tissue meets a predetermined profile among a plurality of observation points having different depth presents, as the analysis target region.

Thus, it is possible to extract the observation point at which the propagation direction of the shear wave and the propagation directions of the shear wave and the observation line at the propagation analysis are substantially perpendicular as the analysis target region.

(7) In addition, in the ultrasound diagnostic apparatus according to (1) to (6) above, the push wave pulse transmitter may continuously transmit a push wave in order of depth to a plurality of transmission focus points having different depth.

Thus, when the shear waves propagating from the plurality of transmission focus points are combined, the shape of the wavefront of the shear wave is close to plane, and the analysis target region becomes large. Therefore, it is possible to perform precise propagation analysis over a wide range.

(8) In addition, in the ultrasound diagnostic apparatus according to (1) to (6) above, the push wave pulse transmitter may select one transmission focus point from a plurality of transmission focus points having different depth and transmits a push wave, the displacement detector may detect the displacement of the tissue at each of some or all observation points in the region of interest on the basis of reflected detection waves received corresponding to the push wave, and push wave transmission by the push wave pulse transmitter and detection of the displacement by the displacement detector may be performed while the transmission focus point is changed to detect the displacement of the tissue at all the observation points in the region of interest.

Thus, when the precision of the displacement detected through detection of the displacement on the basis of the transmission of one-time push wave and the subsequent reflected detection wave is not sufficient enough, the operation is repeated while the transmission focus point is changed. Thus, it is possible to precisely detect the displacement of the tissue at all the observation points in the region of interest.

(9) In addition, the ultrasound diagnostic apparatus according to (1) to (8) above may further include an image outputter that outputs information indicating an elastic modulus of the subject at each of a plurality of observation points present in the analysis target region on the basis of the propagation velocity of the shear wave.

(10) In addition, in the ultrasound diagnostic apparatus according to (9) above, the image outputter may output an elasticity image that indicates information indicating a positional relationship between the plurality of observation points in the region of interest and the elastic modulus of each observation point.

Thus, the distribution of the elastic modulus on the basis of the propagation analysis of the shear wave can be displayed as an image, which is easy to understand.

(11) In addition, in the ultrasound diagnostic apparatus according to (10) above, the propagation information analyzer may further calculate the propagation velocity of the shear wave with regard to an observation point included in the region of interest but not present in the analysis target region, and the image outputter may output information indicating an elastic modulus of the observation point included in the region of interest but not present in the analysis target region, to the elasticity image.

Thus, the elastic modulus can be displayed with regard also to a region where the precision of the propagation analysis of the shear wave is low.

(12) In addition, in the ultrasound diagnostic apparatus according to (9) or (10) above, the analysis target determiner may calculate a parameter indicating steepness of the time change of the displacement of the tissue at the plurality of observation points, and the image outputter may output the parameter, which is superimposed on the elasticity image.

Thus, it is possible to display the level of the precision of the elastic modulus of each observation point.

(13) In addition, in the ultrasound diagnostic apparatus according to (10) above, the analysis target determiner may calculate a parameter indicating steepness of the time change of the displacement of the tissue at the plurality of observation points, and the image outputter may output information indicating an elastic modulus to the elasticity image only with regard to an observation point at which the parameter is equal to or more than a predetermined reference.

Thus, the elastic modulus can be displayed only with regard to the observation point where the precision of the elastic modulus is high among the observation points other than the analysis target region.

(14) In addition, in the ultrasound diagnostic apparatus according to (10) to (13) above, wherein the image outputter outputs a position of the observation point corresponding to the analysis target region, which is superimposed on the elasticity image.

Thus, the position of the observation point where the precision of the elastic modulus is high can be displayed together with the elastic modulus.

The ultrasound diagnostic apparatus and the ultrasound signal processing method according to the present disclosure are useful for tissue hardness measurement using ultrasounds. Therefore, the precision of the tissue hardness measurement can be increased, and the ultrasound diagnostic apparatus and the ultrasound signal processing method according to the present disclosure are highly usable for a medical diagnostic device or the like.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound diagnostic apparatus to which a probe including a plurality of transducers arranged can be connected, causing the probe to transmit a push wave in which ultrasound beams are converged into a subject to detect propagation velocity of a shear wave generated by acoustic radiation pressure of the push wave, the ultrasound diagnostic apparatus comprising:
a push wave pulse transmitter that uses a plurality of transmission transducers selected from the plurality of transducers to transmit a push wave that converges to one or more transmission focus points in the subject;
a detection wave pulse transmitter that supplies a detection wave pulse to some or all of the plurality of transducers to cause the plurality of transducers to transmit, following transmission of the push wave, a detection wave that passes by a region of interest indicating an analysis target range in the subject multiple times;
a displacement detector that detects displacement of a tissue at each of a plurality of observation points in the region of interest on the basis of reflected detection waves received in a time series by the plurality of transducers corresponding to each of detection waves of the multiple times;
an analysis target determiner that determines an analysis target region, that is a target of shear wave propagation analysis, on the basis of steepness of a time change of displacement of the tissue at the plurality of observation points; and
a propagation information analyzer that calculates the propagation velocity of the shear wave at each observation point present in the analysis target region on the basis of displacement of the tissue at the plurality of observation points present in the analysis target region.

2. The ultrasound diagnostic apparatus according to claim 1, wherein
the propagation information analyzer specifies a time when a value of displacement is maximum with regard to each observation point present in the analysis target region and treats the specified time as a time when the shear wave passed by the observation point to calculate velocity of the shear wave.

3. The ultrasound diagnostic apparatus according to claim 1, wherein
the analysis target determiner, on the basis of the time change of the displacement of the tissue at an observation point present at a depth of a predetermined range including a depth at which the transmission focus point is present, selects the analysis target region from the depth of the predetermined range.

4. The ultrasound diagnostic apparatus according to claim 1, wherein
the analysis target determiner, on the basis of the time change of the displacement of the tissue at an observation point present at a depth of a predetermined range including the analysis target region determined on an acoustic line adjacent on a side near the transmission focus point, selects an analysis target region on the acoustic line from the depth of the predetermined region.

5. The ultrasound diagnostic apparatus according to claim 1, wherein
the analysis target determiner determines that an observation point at which the time change of the displacement of the tissue is maximum among a plurality of observation points having different depth is included in the analysis target region.

6. The ultrasound diagnostic apparatus according to claim 1, wherein
the analysis target determiner determines that an observation point at which a profile of the time change of the displacement of the tissue meets a predetermined profile among a plurality of observation points having different depth is included in the analysis target region.

7. The ultrasound diagnostic apparatus according to claim 1, wherein
the push wave pulse transmitter continuously transmits a push wave in order of depth to a plurality of transmission focus points having different depth.

8. The ultrasound diagnostic apparatus according to claim 1, wherein
the push wave pulse transmitter selects one transmission focus point from a plurality of transmission focus points having different depth and transmits a push wave,
the displacement detector detects the displacement of the tissue at each of some or all observation points in the region of interest on the basis of reflected detection waves received corresponding to the push wave, and
push wave transmission by the push wave pulse transmitter and detection of the displacement by the displacement detector are performed while the transmission focus point is changed to detect the displacement of the tissue at all the observation points in the region of interest.

9. The ultrasound diagnostic apparatus according to claim 1, further comprising
an image outputter that outputs information indicating an elastic modulus of the subject at each of a plurality of observation points present in the analysis target region on the basis of the propagation velocity of the shear wave.

10. The ultrasound diagnostic apparatus according to claim 9, wherein
the image outputter outputs an elasticity image that indicates information indicating a positional relationship between the plurality of observation points in the region of interest and the elastic modulus of each observation point.

11. The ultrasound diagnostic apparatus according to claim 10, wherein
the propagation information analyzer further calculates the propagation velocity of the shear wave with regard to an observation point included in the region of interest but not present in the analysis target region, and
the image outputter outputs information indicating an elastic modulus of the observation point included in the region of interest but not present in the analysis target region, to the elasticity image.

12. The ultrasound diagnostic apparatus according to claim 9, wherein
the analysis target determiner calculates a parameter indicating steepness of the time change of the displacement of the tissue at the plurality of observation points, and
the image outputter outputs the parameter, which is superimposed on the elasticity image.

13. The ultrasound diagnostic apparatus according to claim 10, wherein
the analysis target determiner calculates a parameter indicating steepness of the time change of the displacement of the tissue at the plurality of observation points, and
the image outputter outputs information indicating an elastic modulus to the elasticity image only with regard to an observation point at which the parameter is equal to or more than a predetermined reference.

14. The ultrasound diagnostic apparatus according to claim 10, wherein
the image outputter outputs a position of the analysis target region, which is superimposed on the elasticity image.

15. An ultrasound signal processing method using a probe including a plurality of transducers arranged to transmit a push wave in which ultrasound beams are converged into a subject to detect propagation velocity of a shear wave generated by acoustic radiation pressure of the push wave, the ultrasound signal processing method comprising:
by using a plurality of transmission transducers selected from the plurality of transducers, transmitting a push wave that converges to one or more transmission focus points in the subject;
by supplying a detection wave pulse to some or all of the plurality of transducers, causing the plurality of transducers to transmit, following transmission of the push wave, a detection wave that passes by a region of interest indicating an analysis target range in the subject multiple times;
detecting displacement of a tissue at each of a plurality of observation points in the region of interest on the basis of reflected detection waves received in a time series by the plurality of transducers corresponding to each of detection waves of the multiple times;
determining an analysis target region, that is a target of shear wave propagation analysis, on the basis of steepness of a time change of displacement of the tissue at the plurality of observation points; and
calculating the propagation velocity of the shear wave at each observation point present in the analysis target region on the basis of displacement of the tissue at the plurality of observation points present in the analysis target region.
